(19) United States
(12) Patent Application Publication
(10) Pub. No.: US 2022/0136065 A1
(43) Pub. Date: May 5, 2022

(54) HIGH-GRADE SEROUS OVARIAN CARCINOMA (HGSOC)

(71) Applicant: Oxford University Innovation Limited, Oxford (Oxfordshire) (GB)

(72) Inventors: Ahmed Ashour AHMED, Oxford (Oxfordshire) (GB); Zhiyuan HU, Oxford (Oxfordshire) (GB); Christopher YAU, Birmingham (West Midlands) (GB)

(21) Appl. No.: 17/434,474

(22) PCT Filed: Feb. 7, 2020
(86) PCT No.: PCT/GB2020/050286

§ 371 Date: Aug. 27, 2021

(30) Foreign Application Priority Data
Feb. 27, 2019 (GB) 1902653.3

(51) Int. Cl. C12Q 1/6886; G01N 33/50; C12Q 1/6837

(52) U.S. Cl. CPC C12Q 1/6886; G01N 33/5023; C12Q 2600/106; C12Q 2600/112; C12Q 2600/158; C12Q 1/6837

(57) ABSTRACT

The present invention relates to a method of determining the status of high-grade serous ovarian carcinoma (HGSOC) in a subject, the method comprising: providing a sample obtained from the subject; and detecting the presence of HGSOC biomarkers in the sample, wherein the method comprises detecting the presence of: a differentiated cell type; a KRT17 Cluster cell type; an epithelial-mesenchymal transition (EMT) cell type; a cell cycle cell type; and a ciliated cell type; wherein the level of the biomarkers is used to determine the fraction of EMT cells in the high-grade serous ovarian carcinoma in the subject. The invention further relates to associated kits, use and methods of treatment.

The present invention relates to a method of determining the status of high-grade serous ovarian carcinoma (HGSOC) in a subject, related methods of detection, treatments, compositions and kits.

High-grade serous ovarian carcinoma (HGSOC) is a type of tumour that arises from the fallopian tube epithelium and the epithelial layer in the abdominopelvic cavity. HGSOCs make up the majority of ovarian cancer cases and have the lowest survival rates. HGSOC is distinct from low-grade serous ovarian carcinoma (LGSOC). LGSOC is less aggressive and is associated with better survival.

Precise and robust stratification of tumors facilitates diagnosis and treatment. It has been achieved in many cancer types. For example, the subtypes of breast cancer, luminal and basal, have been connected to the cell types of origin. This subtyping has promoted the delivery of efficient treatments. However, for high-grade serous ovarian carcinoma (HGSOC), its classification remains a major challenge due to its genomic complexity. Most of the published molecular classifications were based on the global expression matrix (Bell et al., 2011; Tothill et al., 2008). For instance, the TCGA (The Cancer Genome Atlas) study clustered HGSOC tumors into four distinct groups (immunoreactive, differentiated, proliferative and mesenchymal) based on 1,500 high variance genes. However, a recent consensus clustering study pointed out that this discrete classification system is not robust, and a subset of tumors is unclassifiable (Chen et al., 2018).

Recent evidences strongly support the tubal origin hypothesis that HGSOC originates from the fallopian tube epithelium (FTE) by mouse models and genetic evolutional analysis. FTE is a single-cell layer of secretory cells and ciliated cells, whereas knowledge of these cellular subtypes is limited to few well-known markers, such as PAX8 and TUBB4. Although the HGSOC presumably originates from FTE secretory cells, the classification and understanding of secretory cells remains elusive. Moreover, the “peg” basal cells in FTE have been proposed as stem cells; however, other studies reported infiltrated lymphocytes with similar morphology as those peg cells. Limited understanding regarding of FTE has hindered further investigation into HGSOC.

An aim of the invention is to provide improved methods for determining the status or classifying of high-grade serous ovarian carcinoma (HGSOC) in a subject.

According to a first aspect of the invention, there is provided a method of determining the status of high-grade serous ovarian carcinoma (HGSOC) in a subject, the method comprising:
providing a sample obtained from the subject; and
detecting the presence of HGSOC biomarkers in the sample, wherein the method comprises detecting the presence of:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding epithelial-mesenchymal transition (EMT) biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL;
wherein the level of the biomarkers is used to determine the fraction of EMT cells in the high-grade serous ovarian carcinoma in the subject.

According to another aspect of the invention, there is provided a method of determining the status of high-grade serous ovarian carcinoma (HGSOC) in a subject, the method comprising:
providing a sample obtained from the subject; and
detecting the presence of HGSOC biomarkers in the sample, wherein the method comprises detecting the presence of:
one or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding epithelial-mesenchymal transition (EMT) biomarker proteins selected from the group comprising SPARC, SERPINF1, DCN, SFRP4, CRISPLD2, TIMP3, CNN1, MYH11, MFAP4, ENG, EFEMP1, and RGS16;

one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and

one or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78;
wherein the level of the biomarkers is used to determine the fraction of EMT cells in the high-grade serous ovarian carcinoma in the subject.

According to another aspect of the invention, there is provided a method of determining the status of high-grade serous ovarian carcinoma (HGSOC) in a subject, the method comprising:
providing a sample obtained from the subject; and
detecting the presence of HGSOC biomarkers in the sample, wherein the method comprises detecting the presence of:
one or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding epithelial-mesenchymal transition (EMT) biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78;
wherein the level of the biomarkers is used to determine the fraction of EMT cells in the high-grade serous ovarian carcinoma in the subject.

In one embodiment, the method determines the presence and level of the cell types (e.g. differentiated, KRT17 Cluster, EMT, cell cycle, and ciliated) in the HGSOC by detecting one or more of their representative biomarkers listed herein, for example listed in Table 1 or Table 2, or the combination thereof.

In one embodiment, the fraction of EMT cells in the high-grade serous ovarian carcinoma in the subject is compared to a pre-determined threshold level to indicate if the high-grade serous ovarian carcinoma in the subject is an EMT subclass of high-grade serous ovarian carcinoma.

In one embodiment, the level of the EMT biomarkers/cell types relative to the differentiated, KRT17 Cluster, cell cycle and ciliated biomarkers/cell types is indicative of the fraction of EMT cells, and the fraction of EMT cells above a pre-determined threshold level is indicative of an EMT subclass of high-grade serous ovarian carcinoma in the subject.

In one embodiment, an EMT subclass (also known as an “EMT-high subclass) of HGSOC is indicative of a poorer prognosis for the subject. Additionally or alternatively, an EMT subclass of HGSOC is indicative of an aggressive form of a HGSOC in a subject.

The invention herein advantageously identifies a subclass of patients having EMT- type HGSOC. The hazard of death in EMT tumours as defined by the method of the invention is at least twice that for non-EMT tumours for any given period of time. For example for TCGA data, the hazard risk is determined to be 2.297 (95% Confidence interval=1.291˜4.087). For AOCS data, the hazard risk is determined to be 2.691 (95% Confidence interval=1.556˜4.655).

The hazard risk score from an indication of an EMT subclass of high-grade serous ovarian carcinoma in the subject may be at least 2. In another embodiment, the hazard risk score from an indication of an EMT subclass of high-grade serous ovarian carcinoma in the subject may be at least 2.297. In another embodiment, the hazard risk score from an indication of an EMT subclass of high-grade serous ovarian carcinoma in the subject prognosis may be at least 2.691.

The present invention advantageously identifies cellular subtypes in FTE, which have been thoroughly studied at the transcriptomic level. In particular, the invention profiles the fallopian tube epithelium from patients with HGSOC or endometrium cancer to delineate subtypes in FTE secretory cells and their marker genes. These markers from FTE single cells can then advantageously be used to stratify HSGOCs and identify a tumor subtype with poor overall survival, in particular an EMT subclass of HSGOC with poor overall survival.

According to another aspect of the invention, there is provided a method of detecting a panel of biomarkers in a sample of a subject, the method comprising: providing a sample obtained from a subject and detecting the presence of:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

According to another aspect of the invention, there is provided a method of detecting a panel of biomarkers in a sample of a subject, the method comprising: providing a sample obtained from a subject and detecting the presence of:
one or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

According to another aspect of the invention, there is provided a method of detecting a panel of biomarkers in a sample of a subject, the method comprising: providing a sample obtained from a subject and detecting the presence of:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

According to another aspect of the invention, there is provided a method of detecting a panel of biomarkers in a sample of a subject, the method comprising: providing a sample obtained from a subject and detecting the presence of:
one or more of differentiated cell biomarker proteins and/or nucleic acid encoding differentiated cell biomarker proteins selected from the group comprising

LTBP4, SLC25A25, LAMC2, DHCR24, PLK3, LRG1 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

According to another aspect of the invention, there is provided a method of detecting a panel of biomarkers in a sample of a subject, the method comprising: providing a sample obtained from a subject and detecting the presence of:
one or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

According to another aspect of the invention, there is provided a method of detecting the complexing of a panel of probes with a panel of biomarkers in a sample of a subject, the method comprising:
providing a sample obtained from a subject and detecting the complexing of a panel of probes with:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

According to another aspect of the invention, there is provided a method of detecting the complexing of a panel of probes with a panel of biomarkers in a sample of a subject, the method comprising:
providing a sample obtained from a subject and detecting the complexing of a panel of probes with:
one or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

According to another aspect of the invention, there is provided a method of detecting the complexing of a panel of probes with a panel of biomarkers in a sample of a subject, the method comprising:
providing a sample obtained from a subject and detecting the complexing of a panel of probes with:
one or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

In one embodiment, detecting the presence of a biomarker may comprise the detecting the presence or absence, or the level of the biomarkers. In one embodiment, detecting the presence of a biomarker may comprise the detecting of a level of the biomarker. In one embodiment, detecting the presence or level of a biomarker may comprise determining the expression values for the biomarkers. The pre-determined threshold level may be a pre-determined threshold expression value.

In one embodiment, detecting the complexing of the panel of probes comprises detecting the level of complexing of the panel of probes.

In one embodiment, the level of expression of the biomarkers is indicative of an EMT subclass of high-grade serous ovarian carcinoma in the subject.

The expression value of the biomarkers may be determined using the counts of transcripts to determine the expression such as TCGA data or relative to a standard quantitative or relative transcript data set, such as AOCS data.

In one embodiment, the expression values of the biomarkers that represent the EMT type are compared to the expression values of other types (i.e. differentiated, KRT17 Cluster, cell cycle and ciliated) so that an estimate of the fraction of cells that represent EMT cells is obtained.

The skilled person will be aware of appropriate statistical methods, mathematical methods or computational algorithms, such as standard deconvolution analysis, to estimate the fraction of cells from the expression values. To estimate the fraction of cells from the expression values the level of expression of the EMT biomarkers to those of other tumour types may be compared.

For example, deconvolution analysis may be provided by using the CIBERSORT method (https://cibersort.stanford.edu/), which is an analytical tool developed by Newman et al. (2015. Nature Methods volume 12, pages 453-457, and which is incorporated herein by reference) to provide an estimation of the abundances of member cell types in a mixed cell population, using gene expression data.

In one embodiment, the level of the EMT biomarkers above a pre-determined threshold expression value of the EMT biomarkers is indicative of an EMT subclass of high-grade serous ovarian carcinoma in the subject. In an embodiment using the counts of transcripts (e.g. using TCGA data) to determine the expression value of the EMT biomarkers the pre-determined threshold level of the fraction of EMT cells may be at least 0.2. In another embodiment using a quantitative or relative method (e.g. using AOCS data) to determine the expression value of the EMT biomarkers the pre-determined threshold level of the fraction of EMT cells may be at least 0.4. In one embodiment, the pre-determined threshold level of the fraction of EMT cells may be at least 0.2. In one embodiment, the pre-determined threshold level of the fraction of EMT cells may be at least 0.4.

The method of the invention may be used, for example, for any one or more of the following: to advise on the prognosis for a subject with HGSOC; to advise on treatment options and/or to monitor effectiveness or response of a subject to a treatment for HGSOC. The presence, or level, of the biomarkers may be used to stratify patients. This stratification may be used to decide the appropriate treatment.

Information regarding the HGSOC status of a subject may be relayed to a third party, such as a doctor, other medical professional, pharmacist or other interested party. This information may be relayed digitally, for example via email, SMS or other digital means.

Biomarker Determination/Detection

In one embodiment, the presence, absence, or level of a biomarker may be determined by any suitable assay. In one embodiment, detecting the presence, absence, or level of a biomarker may comprise the use of a probe, such as an oligonucleotide probe. The detecting the presence or level of a biomarker may comprise the detection or measurement of hybridisation of an oligonucleotide to a target sequence of nucleic acid encoding the biomarker. The detecting the presence or level of a biomarker may comprise the detection of binding of a probe to a biomarker, or nucleic acid encoding the biomarker.

The nucleic acid encoding the biomarker may comprise mRNA transcripts or cDNA copies thereof. Therefore the method may comprise determining the transcript level of the biomarkers. The transcript level may be the transcript numbers or the relative levels. In one embodiment, the method comprises the provision of cDNA copies of RNA transcripts of nucleic acid encoding the biomarkers. cDNA copies of all RNA transcript species in the sample may be provided.

The skilled person will recognise there are a number of methods and technologies available to determine the presence and/or level of a biomarker or nucleic acid encoding a biomarker. For example the detection may comprise Northern blot analysis, nuclease protection assays (NPA), in situ hybridization, or reverse transcription-polymerase chain reaction. The detection may comprise the use of Surface-enhanced Raman Spectroscopy (SERS) to detect probes labelled with Raman- active dyes.

The Probes for Detection

In one embodiment, the probes are oligonucleotide probes. An oligonucleotide probe may comprise or consist of a sequence that is substantially or fully complementary to a nucleic acid sequence of a biomarker, or an mRNA or cDNA copy thereof. Each biomarker may be provided with a corresponding oligonucleotide probe that is capable of hybridisation with the sequence encoding the biomarker, for example under stringent conditions. The oligonucleotide probe may comprise or consist of a sequence that is sufficiently complementary to a nucleic acid sequence of a biomarker, or an mRNA or cDNA copy thereof to enable specific hybridisation.

The skilled person will recognise that the number of transcripts of biomarker sequence in a sample or cell can be measured using a variety of techniques.

The oligonucleotide probes may be sufficient length to provide specific hybridisation to a target sequence (of the biomarker) under stringent conditions. In one embodiment, the oligonucleotide probes are at least 10 nucleotides in length. In one embodiment, the oligonucleotide probes are at least 15 nucleotides in length. In one embodiment, the oligonucleotide probes are between 10 and 200 nucleotides in length. In one embodiment, the oligonucleotide probes are between 10 and 100 nucleotides in length. In one embodiment, the oligonucleotide probes are between 10 and 50 nucleotides in length. In one embodiment, the oligonucleotide probes are between 10 and 30 nucleotides in length. In one embodiment, the oligonucleotide probes are between 15 and 30 nucleotides in length.

The oligonucleotide probes may comprise or consist of DNA or RNA. In another embodiment, oligonucleotide probes may comprise or consist of nucleotide analogues, such as PNA, LNA, or PMO; or combinations thereof. The oligonucleotide probes may comprise or consist of DNA and one or more nucleotide analogues, such as PNA, LNA, or PMO.

In one embodiment the oligonucleotide probes are reporter probes, for example that provide a signal upon hybridisation (directly or indirectly via another molecule). The probe may be labelled, for example with a fluorescent marker or dye, or a radiolabel. Fluorescent dyes may comprise one or more of coumarin, Cy2, Cy3, rhodamine red, texas red, Cy5, Cy5.5 and Cy7, or functional equivalents or derivatives thereof. The label may comprise a Raman-active dye, such as Azo dyes. Examples of Raman-active dyes that may be used are Rhodamine 6G, Cy3, Cy5, or Malachite Green. The probe may be labelled with a fluorescent barcode (e.g. a series of different fluorescent molecules that can be used to identify a probe by determining the sequence or position of the fluorescent molecules in the fluorescent barcode. In another embodiment the label may be a genetic barcode (i.e. a sequence of nucleotides that can be used to label the probe).

In one embodiment, the oligonucleotide probes are immobilised on a substrate. In one embodiment, the oligonucleotide probes comprise a tag for capture/anchoring (or otherwise known as immobilising) on a substrate. The tag on the probe may comprise a biotin-avidin tag, e.g. the probe may be biotinylated. The tag on the probe may comprise a nanoparticle, such as a metal nanoparticle. In another embodiment the tag may be a nucleic acid sequence of the probe, which is capable of hybridising to a complementary sequence of a nucleic acid that is anchored to a substrate.

The detection of the biomarkers may comprise the use of nCounter® Technology (otherwise known as direct multiplexed measurement of gene expression with color-coded probe pairs) (e.g. by Nanostring™) (see Geiss et al. 2008 (Direct multiplexed measurement of gene expression with color-coded probe pairs. Nature Biotechnology volume 26, pages 317-325), which is herein incorporated by reference). In one embodiment, the probe is a capture probe that is capable of hybridizing to a target nucleic acid sequence of the biomarker sequence, or a mRNA or cDNA copy thereof. The capture probe may comprise an anchor tag that is capable of anchoring the capture probe to a substrate. A second probe, known as a reporter probe, may be provided to further hybridise to the target sequence, or mRNA or cDNA copy thereof, together with the capture probe and form a target-probe complex. The reporter probe may comprise a fluorescent barcode for detection and identification of the target-probe complex. The target-probe complexes may be isolated by capture using the tag on the capture probe, and excess probes may be removed, including excess reporter probes and/or excess non-hybridized capture probes. The target-probe complexes may be detected in a device capable of imaging and reading fluorescent barcodes of the target-probe complexes.

In another embodiment, the probes may be PCR primers, such that the biomarker may be detected by PCR amplifying nucleic acid encoding the biomarker. In an embodiment where the probes are primers, a forward and reverse primer may be provided. The skilled person will readily be able to design and provide appropriate primers for a given biomarker nucleic acid encoding sequence. The PCR may be RT-PCR.

The probes may be provided in the form of a microarray.

In one embodiment, polypeptides of the biomarkers are detected/measured. In one embodiment, polypeptides of the biomarkers are detected by antibodies, for example by an Immunohistochemistry (IHC) panel of biomarkers. The antibodies may be monoclonal. The antibodies may be immobilised on a substrate. The antibodies may be conjugated to a label, such as an enzyme (such as peroxidase) or fluorophore.

Sequencing

In one embodiment, detecting the biomarkers may comprise the detection by sequencing nucleic acid encoding the biomarkers, such as mRNA/transcriptome sequencing. The sequencing may comprise single cell RNA transcriptome sequencing. The sequence may comprise single molecule sequencing, such as nanopore sequencing.

Biomarkers for Detection

Table 1 lists a first panel of 52 biomarkers that have been found to enable the sub-classification of HGSOCs in accordance with the invention. Table 2 lists a second panel of 52 biomarkers that have been found to enable the sub-classification of HGSOCs in accordance with the invention. In one embodiment, all 52 biomarkers of Table 1 may be detected/probed. In another embodiment, all 52 biomarkers of Table 2 may be detected/probed. In another embodiment, all biomarkers of Table 1 and 2 may be detected/probed. In one embodiment, at least 50%, 60%, 70% 90%, 95%, or 98% of the 52 biomarkers of Table 1 may be detected/probed. In another embodiment, at least 50%, 60%, 70% 90%, 95%, or 98% of the 52 biomarkers of Table 2 may be detected/probed. In one embodiment, at least one or two biomarkers from each signature group (i.e. differentiated, KRT17 Cluster, EMT, cell cycle, and ciliated) of Table 1 may be detected. In another embodiment, at least one or two biomarkers from each signature group (i.e. differentiated, KRT17 Cluster, EMT, cell cycle, and ciliated) of Table 2 may be detected. In another embodiment, at least one or two biomarkers from each signature group (i.e. differentiated, KRT17 Cluster, EMT, cell cycle, and ciliated) of a combination of Tables 1 and 2 may be detected. In another embodiment, at least 50% of the biomarkers from each signature group (i.e. differentiated, KRT17 Cluster, EMT, cell cycle, and ciliated) of Table 1 or Table 2 may be detected. In another embodiment, at least 50% of the biomarkers from each signature group (i.e. differentiated, KRT17 Cluster, EMT, cell cycle, and ciliated) of Table 1 and Table 2 in combination may be detected. In another embodiment, at least 60% or 80% of the biomarkers from each signature group (i.e. differentiated, KRT17 Cluster, EMT, cell cycle, and ciliated) of Table 1 may be detected. In another embodiment, at least 60% or 80% of the biomarkers from each signature group (i.e. differentiated, KRT17 Cluster, EMT, cell cycle, and ciliated) of Table 2 may be detected. In another embodiment, at least 60% or 80% of the biomarkers from each signature group (i.e. differentiated, KRT17 Cluster, EMT, cell cycle, and ciliated) of Table 1 and Table 2 combined may be detected. The skilled person will recognise that reference to the combined biomarkers of Tables 1 and 2 comprises duplicates, which may only be counted once as a single biomarker.

TABLE 1
first panel list of identified biomarkers

| | Gene. | | | Entrez_ |
|---|---|---|---|---|
| ID | Symbol | Signature | Ensembl_gene_id | gene_id |
| 1 | LTBP4 | Differentiated | ENSG00000090006 | 8425 |
| 2 | PTGS1 | Differentiated | ENSG00000095303 | 5742 |
| 3 | SLC25A25 | Differentiated | ENSG00000148339 | 114789 |
| 4 | LAMC2 | Differentiated | ENSG00000058085 | 3918 |
| 5 | LRG1 | Differentiated | ENSG00000171236 | 116844 |
| 6 | DHCR24 | Differentiated | ENSG00000116133 | 1718 |
| 7 | LDLR | Differentiated | ENSG00000130164 | 3949 |
| 8 | SPP1 | KRT17 Cluster | ENSG00000118785 | 6696 |
| 9 | IL1B | KRT17 Cluster | ENSG00000125538 | 3553 |
| 10 | IL1RN | KRT17 Cluster | ENSG00000136689 | 3557 |
| 11 | KRT23 | KRT17 Cluster | ENSG00000108244 | 25984 |
| 12 | ALDH3B2 | KRT17 Cluster | ENSG00000132746 | 222 |
| 13 | SUSD2 | KRT17 Cluster | ENSG00000099994 | 56241 |
| 14 | DEFB1 | KRT17 Cluster | ENSG00000164825 | 1672 |
| 15 | HLA-DQA2 | KRT17 Cluster | ENSG00000237541 | 3118 |
| 16 | CYP4B1 | KRT17 Cluster | ENSG00000142973 | 1580 |
| 17 | PIGR | KRT17 Cluster | ENSG00000162896 | 5284 |
| 18 | SPARC | EMT | ENSG00000113140 | 6678 |
| 19 | SERPINF1 | EMT | ENSG00000132386 | 5176 |
| 20 | DCN | EMT | ENSG00000011465 | 1634 |
| 21 | SFRP4 | EMT | ENSG00000106483 | 6424 |
| 22 | CRISPLD2 | EMT | ENSG00000103196 | 83716 |
| 23 | TIMP3 | EMT | ENSG00000100234 | 7078 |
| 24 | CNN1 | EMT | ENSG00000130176 | 1264 |
| 25 | MYH11 | EMT | ENSG00000133392 | 4629 |
| 26 | MFAP4 | EMT | ENSG00000166482 | 4239 |
| 27 | ENG | EMT | ENSG00000106991 | 2022 |
| 28 | EFEMP1 | EMT | ENSG00000115380 | 2202 |
| 29 | RGS16 | EMT | ENSG00000143333 | 6004 |
| 30 | FEN1 | Cell cycle | ENSG00000168496 | 2237 |
| 31 | NUSAP1 | Cell cycle | ENSG00000137804 | 51203 |
| 32 | UBE2C | Cell cycle | ENSG00000175063 | 11065 |
| 33 | ZWINT | Cell cycle | ENSG00000122952 | 11130 |
| 34 | PRC1 | Cell cycle | ENSG00000198901 | 9055 |
| 35 | ASF1B | Cell cycle | ENSG00000105011 | 55723 |
| 36 | MCM4 | Cell cycle | ENSG00000104738 | 4173 |
| 37 | GINS2 | Cell cycle | ENSG00000131153 | 51659 |
| 38 | CENPM | Cell cycle | ENSG00000100162 | 79019 |
| 39 | MCM2 | Cell cycle | ENSG00000073111 | 4171 |
| 40 | TK1 | Cell cycle | ENSG00000167900 | 7083 |
| 41 | MCM6 | Cell cycle | ENSG00000076003 | 4175 |
| 42 | SMC4 | Cell cycle | ENSG00000113810 | 10051 |
| 43 | CENPU | Cell cycle | ENSG00000151725 | 79682 |
| | (MLF1IP) | | | |
| 44 | MAD2L1 | Cell cycle | ENSG00000164109 | 4085 |
| 45 | TEKT1 | Ciliated | ENSG00000167858 | 83659 |
| 46 | FAM92B | Ciliated | ENSG00000153789 | 339145 |
| 47 | SNTN | Ciliated | ENSG00000188817 | 132203 |
| 48 | LRRC46 | Ciliated | ENSG00000141294 | 90506 |
| 49 | EFCAB1 | Ciliated | ENSG00000034239 | 79645 |
| 50 | CDHR3 | Ciliated | ENSG00000128536 | 222256 |
| 51 | C6orf118 | Ciliated | ENSG00000112539 | 168090 |
| 52 | CCDC78 | Ciliated | ENSG00000162004 | 124093 |

TABLE 2
second panel list of identified biomarkers

| | | | Entrez_ | |
|---|---|---|---|---|
| ID | HGNC_symbol | Signature | gene_id | Ensembl_gene_id |
| 1 | LTBP4 | Differentiated | 8425 | ENSG00000090006 |
| 2 | SLC25A25 | Differentiated | 114789 | ENSG00000148339 |
| 3 | LAMC2 | Differentiated | 3918 | ENSG00000058085 |
| 4 | DHCR24 | Differentiated | 1718 | ENSG00000116133 |
| 5 | PLK3 | Differentiated | 1263 | ENSG00000173846 |
| 6 | LRG1 | Differentiated | 116844 | ENSG00000171236 |
| 7 | LDLR | Differentiated | 3949 | ENSG00000130164 |
| 8 | SPP1 | KRT17 Cluster | 6696 | ENSG00000118785 |
| 9 | IL1B | KRT17 Cluster | 3553 | ENSG00000125538 |
| 10 | IL1RN | KRT17 Cluster | 3557 | ENSG00000136689 |
| 11 | KRT23 | KRT17 Cluster | 25984 | ENSG00000108244 |
| 12 | ALDH3B2 | KRT17 Cluster | 222 | ENSG00000132746 |
| 13 | SUSD2 | KRT17 Cluster | 56241 | ENSG00000099994 |
| 14 | DEFB1 | KRT17 Cluster | 1672 | ENSG00000164825 |
| 15 | HLA-DQA2 | KRT17 Cluster | 3118 | ENSG00000237541 |
| 16 | CYP4B1 | KRT17 Cluster | 1580 | ENSG00000142973 |
| 17 | PIGR | KRT17 Cluster | 5284 | ENSG00000162896 |
| 18 | SPARC | EMT | 6678 | ENSG00000113140 |
| 19 | SERPINF1 | EMT | 5176 | ENSG00000132386 |
| 20 | DCN | EMT | 1634 | ENSG00000011465 |
| 21 | SFRP4 | EMT | 6424 | ENSG00000106483 |
| 22 | CRISPLD2 | EMT | 83716 | ENSG00000103196 |
| 23 | TIMP3 | EMT | 7078 | ENSG00000100234 |
| 24 | CNN1 | EMT | 1264 | ENSG00000130176 |
| 25 | MYH11 | EMT | 4629 | ENSG00000133392 |
| 26 | MFAP4 | EMT | 4239 | ENSG00000166482 |
| 27 | ENG | EMT | 2022 | ENSG00000106991 |
| 28 | EFEMP1 | EMT | 2202 | ENSG00000115380 |
| 29 | RGS16 | EMT | 6004 | ENSG00000143333 |
| 30 | FEN1 | Cell cycle | 2237 | ENSG00000168496 |
| 31 | NUSAP1 | Cell cycle | 51203 | ENSG00000137804 |
| 32 | UBE2C | Cell cycle | 11065 | ENSG00000175063 |
| 33 | ZWINT | Cell cycle | 11130 | ENSG00000122952 |
| 34 | PRC1 | Cell cycle | 9055 | ENSG00000198901 |
| 35 | ASF1B | Cell cycle | 55723 | ENSG00000105011 |
| 36 | MCM4 | Cell cycle | 4173 | ENSG00000104738 |
| 37 | GINS2 | Cell cycle | 51659 | ENSG00000131153 |
| 38 | CENPM | Cell cycle | 79019 | ENSG00000100162 |
| 39 | MCM2 | Cell cycle | 4171 | ENSG00000073111 |
| 40 | TK1 | Cell cycle | 7083 | ENSG00000167900 |
| 41 | MCM6 | Cell cycle | 4175 | ENSG00000076003 |
| 42 | SMC4 | Cell cycle | 10051 | ENSG00000113810 |
| 43 | CENPU | Cell cycle | 79682 | ENSG00000151725 |
| | (MLF1IP) | | | |
| 44 | MAD2L1 | Cell cycle | 4085 | ENSG00000164109 |
| 45 | TEKT1 | Ciliated | 83659 | ENSG00000167858 |
| 46 | TUBA4B | Ciliated | 80086 | ENSG00000243910 |
| 47 | C20orf85 | Ciliated | 128602 | ENSG00000124237 |
| 48 | CAPSL | Ciliated | 133690 | ENSG00000152611 |
| 49 | LRRC46 | Ciliated | 90506 | ENSG00000141294 |
| 50 | EFCAB1 | Ciliated | 79645 | ENSG00000034239 |
| 51 | C6orf118 | Ciliated | 168090 | ENSG00000112539 |
| 52 | CCDC78 | Ciliated | 124093 | ENSG00000162004 |

In one embodiment, the method comprises detecting the presence or level of:
two or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
two or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
two or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
two or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
two or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

In one embodiment, the method comprises detecting the presence or level of:
two or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
two or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
two or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
two or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
two or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

In one embodiment, the method comprises detecting the presence or level of:
two or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
two or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
two or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
two or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
two or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

In one embodiment, the method comprises detecting the presence or level of:
three or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
three or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
three or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
three or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
three or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

In one embodiment, the method comprises detecting the presence or level of:
three or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
three or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
three or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
three or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
three or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

In one embodiment, the method comprises detecting the presence or level of:
three or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
three or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
three or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
three or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
three or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

In one embodiment, the method comprises detecting the presence or level of:
four or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
four or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
four or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
four or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
four or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

In one embodiment, the method comprises detecting the presence or level of:
four or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
four or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
four or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
four or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
four or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

In one embodiment, the method comprises detecting the presence or level of:
four or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
four or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
four or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
four or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
four or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

In one embodiment, the method comprises detecting the presence or level of:
five or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
5, 6, 7, 8, 9 or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
5, 6, 7, 8, 9, 10, or 11 or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
5, 6, 7, 8, 9, 10, 11, 12, 13, or 14 or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
5, 6, or 7 or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

In one embodiment, the method comprises detecting the presence or level of:
five or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
5, 6, 7, 8, 9 or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
5, 6, 7, 8, 9, 10, or 11 or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
5, 6, 7, 8, 9, 10, 11, 12, 13, or 14 or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
5, 6, or 7 or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

In one embodiment, the method comprises detecting the presence or level of:
five or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
5, 6, 7, 8, 9 or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
5, 6, 7, 8, 9, 10, or 11 or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
5, 6, 7, 8, 9, 10, 11, 12, 13, or 14 or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
5, 6, or 7 or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

In one embodiment, the method comprises detecting the presence or level of:
differentiated cell biomarker proteins and/or nucleic acid encoding differentiated cell biomarker proteins of LTBP4, PTGS1, SLC25A25, LAMC2, LRG1, DHCR24, PLK3 and LDLR;
KRT17 Cluster cell biomarker proteins and/or nucleic acid encoding KRT17 Cluster cell biomarker proteins of SPP1, IL1B, IL1RN, KRT23, ALDH3B2, SUSD2, DEFB1, HLA-DQA2, CYP4B1, and PIGR;
EMT biomarker proteins and/or nucleic acid encoding EMT biomarker proteins of SPARC, SERPINF1, DCN, SFRP4, CRISPLD2, TIMP3, CNN1, MYH11, MFAP4, ENG, EFEMP1, and RGS16;

cell cycle biomarker proteins and/or nucleic acid encoding cell cycle biomarker proteins of FEN1, NUSAP1, UBE2C, ZWINT, PRC1, ASF1B, MCM4, GINS2, CENPM, MCM2, TK1, MCM6, SMC4, CENPU, and MAD2L1; and ciliated cell biomarker proteins and/or nucleic acid encoding ciliated cell biomarker proteins of TEKT1, FAM92B, SNTN, LRRC46, EFCAB1, CDHR3, C6orf118, CCDC78, TUBA4B, C20orf85 and CAPSL.

In one embodiment, the method comprises detecting the presence or level of:
differentiated cell biomarker proteins and/or nucleic acid encoding differentiated cell biomarker proteins of LTBP4, SLC25A25, LAMC2, DHCR24, PLK3, LRG1 and LDLR;
KRT17 Cluster cell biomarker proteins and/or nucleic acid encoding KRT17 Cluster cell biomarker proteins of SPP1, IL1B, IL1RN, KRT23, ALDH3B2, SUSD2, DEFB1, HLA-DQA2, CYP4B1, and PIGR;
EMT biomarker proteins and/or nucleic acid encoding EMT biomarker proteins of SPARC, SERPINF1, DCN, SFRP4, CRISPLD2, TIMP3, CNN1, MYH11, MFAP4, ENG, EFEMP1, and RGS16;
cell cycle biomarker proteins and/or nucleic acid encoding cell cycle biomarker proteins of FEN1, NUSAP1, UBE2C, ZWINT, PRC1, ASF1B, MCM4, GINS2, CENPM, MCM2, TK1, MCM6, SMC4, CENPU, and MAD2L1; and ciliated cell biomarker proteins and/or nucleic acid encoding ciliated cell biomarker proteins of TEKT1, TUBA4B, C20orf85, CAPSL, LRRC46, EFCAB1, C6orf118, and CCDC78.

In one embodiment, the method comprises detecting the presence or level of:
differentiated cell biomarker proteins and/or nucleic acid encoding differentiated cell biomarker proteins of LTBP4, PTGS1, SLC25A25, LAMC2, LRG1, DHCR24, and LDLR;
KRT17 Cluster cell biomarker proteins and/or nucleic acid encoding KRT17 Cluster cell biomarker proteins of SPP1, IL1B, IL1RN, KRT23, ALDH3B2, SUSD2, DEFB1, HLA-DQA2, CYP4B1, and PIGR;
EMT biomarker proteins and/or nucleic acid encoding EMT biomarker proteins of SPARC, SERPINF1, DCN, SFRP4, CRISPLD2, TIMP3, CNN1, MYH11, MFAP4, ENG, EFEMP1, and RGS16;
cell cycle biomarker proteins and/or nucleic acid encoding cell cycle biomarker proteins of FEN1, NUSAP1, UBE2C, ZWINT, PRC1, ASF1B, MCM4, GINS2, CENPM, MCM2, TK1, MCM6, SMC4, CENPU, and MAD2L1; and
ciliated cell biomarker proteins and/or nucleic acid encoding ciliated cell biomarker proteins of TEKT1, FAM92B, SNTN, LRRC46, EFCAB1, CDHR3, C6orf118, and CCDC78.

In one embodiment, the detection does not comprise the detection of immune genes, such as CD8, CD4, CD3 or CD45.

Advantageously, the exclusion of immune genes provides that the classification results are not confounded by the infiltration of lymphocytes.

The Sample

The sample from the subject may be a tissue sample. The tissue sample may comprise or consist of an ovarian cancer biopsy tissue. The ovarian cancer biopsy tissue may be from sites such as the ovary, peritoneum, omentum, or diaphragm. The tissue sample may be a post-operative sample (i.e. a sample taken during surgery on the subject). In another embodiment the sample is a blood or serum sample for the detection of circulating biomarkers.

Providing a sample obtained from a subject may comprises obtaining a tissue sample, such as conducting a biopsy. In another embodiment, the sample may be provided for testing, for example from a third party or from a separate procedure. In one embodiment, the tissue sample is obtained from a biopsy of the tissue. The sample may be a fresh sample (e.g. not frozen or otherwise stored for a period of greater than 1 day), or it may have been frozen, for example for storing the sample, prior to the detection. In another embodiment, the sample may have been preserved or fixed prior to the detection.

The amount of sample may be an amount that provides sufficient biomarker to be measured, for example an amount that provides 1, 2, 3 or more nanograms of RNA.

Some or all of the steps of the method of the invention may be carried out in vitro.

The Subject

The subject may have or is suspected of having ovarian cancer. The subject may have or is suspected of having high-grade serous ovarian cancer.

In one embodiment, the subject is mammalian, such as a human. In one embodiment, the subject is a human adult female.

Other Aspects of the Invention

According to another aspect of the invention, there is provided a composition comprising a panel of probes, wherein the probes are for detecting:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1,

UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and

CAPSL.

According to another aspect of the invention, there is provided a composition comprising a panel of probes, wherein the probes are for detecting:
one or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

According to another aspect of the invention, there is provided a composition comprising a panel of probes, wherein the probes are for detecting:
one or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

According to another aspect of the invention, there is provided a kit for determining the status of high-grade serous ovarian carcinoma (HGSOC) in a subject, the kit comprising a panel of probes, wherein the probes are for detecting:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

According to another aspect of the invention, there is provided a kit for determining the status of high-grade serous ovarian carcinoma (HGSOC) in a subject, the kit comprising a panel of probes, wherein the probes are for detecting:
one or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, TUBA4B, C20orf85, CAPSL, LRRC46, EFCAB1, C6orf118, and CCDC78.

According to another aspect of the invention, there is provided a kit for determining the status of high-grade serous ovarian carcinoma (HGSOC) in a subject, the kit comprising a panel of probes, wherein the probes are for detecting:
one or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

In one embodiment of the composition or the kit, a probe may be provided for each of the 52 biomarkers of Table 1 or Table 2, or a combination of Tables 1 and 2. In one embodiment of the composition or the kit, probes may be provided for at least 50%, 60%, 70% 90%, 95%, or 98% of the 52 biomarkers of Table 1. In another embodiment of the composition or the kit, probes may be provided for at least 50%, 60%, 70% 90%, 95%, or 98% of the 52 biomarkers of Table 2. In another embodiment of the composition or the kit, probes may be provided for at least 50%, 60%, 70% 90%, 95%, or 98% of the biomarkers of Table 1 and Table 2 combined. In one embodiment of the composition or the kit, probes may be provided for at least one or two biomarkers from each signature group (i.e. differentiated, KRT17 Cluster, EMT, cell cycle, and ciliated) of Table 1. In another embodiment of the composition or the kit, probes may be provided for at least one or two biomarkers from each signature group (i.e. differentiated, KRT17 Cluster, EMT, cell cycle, and ciliated) of Table 2. In another embodiment of the composition or the kit, probes may be provided for at least one or two biomarkers from each signature group (i.e. differentiated, KRT17 Cluster, EMT, cell cycle, and ciliated) of Table 1 and Table 2 combined. In another embodiment of the composition or the kit, probes may be provided for at least 50% of the biomarkers from each signature group (i.e. differentiated, KRT17 Cluster, EMT, cell cycle, and ciliated) of Table 1 or Table 2, or Table 1 and Table 2 combined. In another embodiment of the composition or the kit, probes may be provided for at least 60% or 80% of the biomarkers from each signature group (i.e. differentiated, KRT17 Cluster, EMT, cell cycle, and ciliated) of Table 1. In another embodiment of the composition or the kit, probes may be provided for at least 60% or 80% of the biomarkers from each signature group (i.e. differentiated, KRT17 Cluster, EMT, cell cycle, and ciliated) of Table 2. In another embodiment of the composition or the kit, probes may be provided for at least 60% or 80% of the biomarkers from each signature group (i.e. differentiated, KRT17 Cluster, EMT, cell cycle, and ciliated) of Table 1 and Table 2 combined.

According to another aspect of the invention, there is provided a method of selecting a patient for treatment with an agent, agent combination, or composition for treatment or prevention of HGSOC, the method comprising determining the status of high-grade serous ovarian carcinoma (HGSOC) in a subject according to the method if the invention herein, wherein the determination of an EMT subclass of HGSOC indicates that the subject should or should not receive the agent, agent combination, or composition.

The subject may be provided with a choice of not receiving treatment (or advised not to receive treatment) with a therapeutic agent, such as Carboplatin, Paclitaxel or

PARP inhibitors, which may be less effective for EMT HGSOC subjects. PARP inhibitors may comprise Olaparib, Rucaparib, Niraparib, or Talazoparib.

The agent, agent combination, or composition may comprise a therapeutically effective amount of the agent, agent combination, or composition. The agent, agent combination, or composition may be a known to treat HGSOC or EMT HGSOC, or reduce symptoms thereof. The agent may comprise a PI3K pathway inhibitor.

In one embodiment, the treatment comprises immunotherapy. Therefore, the agent may comprise an immunotherapeutic agent. The agent may comprise a vaccine arranged to produce an immune response to HGSOC tumour cells, an antibody, such as a monoclonal antibody, or cell, such as a T-cell, arranged to target HGSOC tumour cells (or markers thereof). The immunotherapy/immunotherapeutic agent may be used in combination with other therapeutic agents, such as PI3K pathway inhibitor.

According to another aspect of the invention, there is provided a PI3K pathway inhibitor for use in the treatment of high-grade serous ovarian carcinoma (HGSOC) in a subject, wherein the treatment comprises selecting the patient for treatment based on the determination of an EMT subclass of high-grade serous ovarian carcinoma in the subject.

According to another aspect of the invention, there is provided an immunotherapeutic agent for use in the treatment of high-grade serous ovarian carcinoma (HGSOC) in a subject, wherein the treatment comprises selecting the patient for treatment based on the determination of an EMT subclass of high-grade serous ovarian carcinoma in the subject.

According to another aspect of the invention, there is provided a method of treating a subject with a PI3K pathway inhibitor, wherein the subject is determined to have an EMT subclass of high-grade serous ovarian carcinoma;
wherein the method of treatment comprises administrating a PI3K pathway inhibitor to the subject.

According to another aspect of the invention, there is provided a method of treating a subject with immunotherapy targeting an EMT subclass of high-grade serous ovarian carcinoma, wherein the subject is determined to have an EMT subclass of high-grade serous ovarian carcinoma;
wherein the method of treatment comprises administrating an immunotherapeutic agent to the subject.

The determination of an EMT subclass of high-grade serous ovarian carcinoma in the subject may comprise the method of determining the status of high-grade serous ovarian carcinoma (HGSOC) of a subject in accordance with the invention herein.

According to another aspect of the invention, there is provided a method of treating a subject with a PI3K pathway inhibitor, wherein the subject has, or is suspected of having, HGSOC,
the method comprising the steps of:
receiving results of a biomarker assay of a tissue sample from the subject to determine if the patient has an EMT subclass of high-grade serous ovarian carcinoma; and
if the subject has an EMT subclass of high-grade serous ovarian carcinoma, then administrating a PI3K pathway inhibitor to the subject.

According to another aspect of the invention, there is provided a method of treating a subject with an immunotherapy targeting HGSOC, wherein the subject has, or is suspected of having, HGSOC,
the method comprising the steps of:
receiving results of a biomarker assay of a tissue sample from the subject to determine if the patient has an EMT subclass of high-grade serous ovarian carcinoma; and
if the subject has an EMT subclass of high-grade serous ovarian carcinoma, then administrating immunotherapeutic agent to the subject.

The biomarker assay may be obtained from conducting the method of determining the status of high-grade serous ovarian carcinoma (HGSOC) of a subject in accordance with the invention herein.

The PI3K pathway inhibitor may comprise a PI3K inhibitor or an AKT inhibitor, or a combination thereof.

In one embodiment, the PI3K inhibitor comprises an PI3K inhibitor selected from pilaralisib, ACP-319 (Acerta Pharma BV), ACP-319 (Acerta Pharma BV), BAY-1082439 (Bayer AG), AZD-8154 (AstraZeneca Plc), BPS-001 (Biopep Solutions Inc), PF-4989216 (Pfizer Inc), BR-101801 (Boryung Pharmaceutical Co Ltd), ZSTK-474 (Zenyaku Kogyo Co Ltd), ZSTK-474 (Zenyaku Kogyo Co Ltd), ZSTK-474 (Zenyaku Kogyo Co Ltd), WX-008 (Chia Tai Tianqing Pharmaceutical Group Co Ltd), IBL-202 (Inflection Biosciences Ltd) IBL-202 (Inflection Biosciences Ltd), SRX-3207 (SignalRx Pharmaceuticals Inc), AMXI-9001 (AtlasMedx Inc), X-480 (Xcovery Holding Company LLC), pictilisib, CLR-457 (Novartis AG), AMG-511 (Amgen Inc), AS-605240 (Merck KGaA), CU-903 (Curis Inc), PI-3065 (F. Hoffmann-La Roche Ltd), acalisib, AEZS-129 (Aeterna Zentaris Inc), GSK-1059615 (GlaxoSmithKline Plc), WX-037 (Heidelberg Pharma AG), and AEZS-132 (Aeterna Zentaris Inc); or combinations thereof.

In one embodiment, the AKT inhibitor comprises an AKT inhibitor or AKT inhibitor combination selected from ipatasertib, LY-2503029 (Eli Lily and Co), capivasertib, MK-2206 (Merck & Co Inc), MK-2206 + selumetinib sulfate, uprosertib, TAS-117 (Taiho Pharmaceutical Co Ltd.), ARQ-751 (ArQule Inc); FXY-1 (Krisani Bio Sciences Pvt Ltd), perifosine, RX-0183 (Rexahn Pharmaceuticals Inc), VLI-27 (NovaLead Pharma Pvt Ltd), PX-316 (Seattle Genetics Inc), J-9 (Columbia University), and afuresertib+trametinib; or combinations thereof.

The immunotherapy may comprise the administration or use of a vaccine arranged to produce an immune response to HGSOC tumour cells, an antibody, such as a monoclonal antibody, or cell, such as a T-cell, arranged to target HGSOC tumour cells. An immunotherapeutic agent may be used or administered in combination with another therapeutic agent, such a PI3K pathway inhibitor.

The method of the invention herein may further comprise a second/follow-up determination of the status of high-grade serous ovarian carcinoma (HGSOC) of a subject. Two or more measurements may be provided to measure the progression of a subject's cancer. For example, a subject's status may be measured two or more times at different time points (for example before, during and after treatment) in order to determining if the EMT HGSOC is reduced or eliminated, or if HGSOC has developed into EMT HGSOC.

According to another aspect of the invention, there is provided the use of a panel of biomarkers for determining the fraction of EMT cells present in HGSOC or determining the status of HGSOC, wherein the biomarkers comprise:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

According to another aspect of the invention, there is provided the use of a panel of biomarkers for determining the fraction of EMT cells present in HGSOC or determining the status of HGSOC, wherein the biomarkers comprise:
one or more of differentiated cell biomarker proteins and/or nucleic acid encoding differentiated cell biomarker proteins selected from the group comprising LTBP4, SLC25A25, LAMC2, DHCR24, PLK3, LRG1 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising EKT1, TUBA4B, C20orf85, CAPSL, LRRC46, EFCAB1, C6orf118, and CCDC78.

According to another aspect of the invention, there is provided the use of a panel of biomarkers for determining the fraction of EMT cells present in HGSOC or determining the status of HGSOC, wherein the biomarkers comprise:
one or more of differentiated cell biomarker proteins and/or nucleic acid encoding differentiated cell biomarker proteins selected from the group comprising LTBP4, PTGS1, SLC25A25, LAMC2, LRG1, DHCR24, and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, and CCDC78.

The use of the biomarkers may comprise the determination of the biomarkers in a sample from a subject, such as a tissue sample.

Definitions

The biomarkers listed herein may include variants of the biomarkers, for example variants having natural mutations/polymorphisms in a population. It is understood that reference to protein or nucleic acid “variants”, it is understood to mean a protein or nucleic acid sequence that has at least 70%, 80%, 90%, 95%, 98%, 99%, 99.9% identity with the sequence of the fore mentioned protein or nucleic acid. The percentage identity may be calculated under standard NCBI blast p/n alignment parameters. “Variants” may also include truncations of a protein or nucleic acid sequence. Variants may include biomarker listed herein comprising the same sequence, but comprising or consisting of 1, 2, 3, 4, 5, 6, 7, 8, 9, or 10, or more modifications, such as substitutions, deletions, additions of nucleotides or bases. Variants may also comprise redundant/degenerate codon variations.

The term “encoding”, may mean at least partially encoding or fully encoding a given biomarker/gene. The at least partially encoding may be at least enough sequence to identify the biomarker/gene.

The term “detected” or “detecting” in the context of a biomarker is understood to mean that an attempt is made to detect the biomarker, such that a biomarker in the sample is attempted to be detected even in the event that the biomarker not present in the sample.

By an “agent” we include all chemical entities, for example oligonucleotides, polynucleotide, polypeptides, peptidomimetics and small compounds.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

Reference to hybridization of a probe or primer herein is understood to mean the specific complementary binding of one molecule to another under standard stringent conditions.

The skilled person will understand that optional features of one embodiment or aspect of the invention may be applicable, where appropriate, to other embodiments or aspects of the invention.

Embodiments of the invention will now be described in more detail, by way of example only, with reference to the accompanying figures.

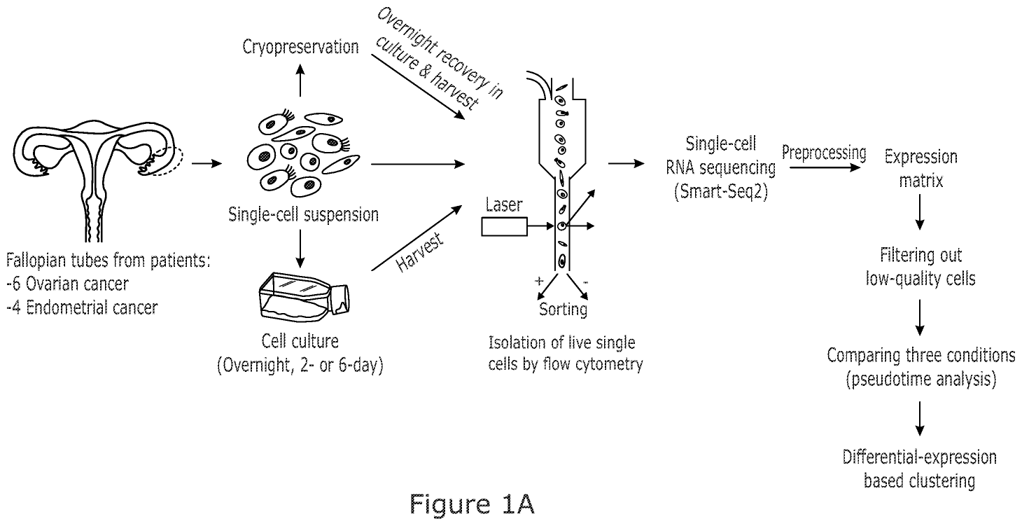

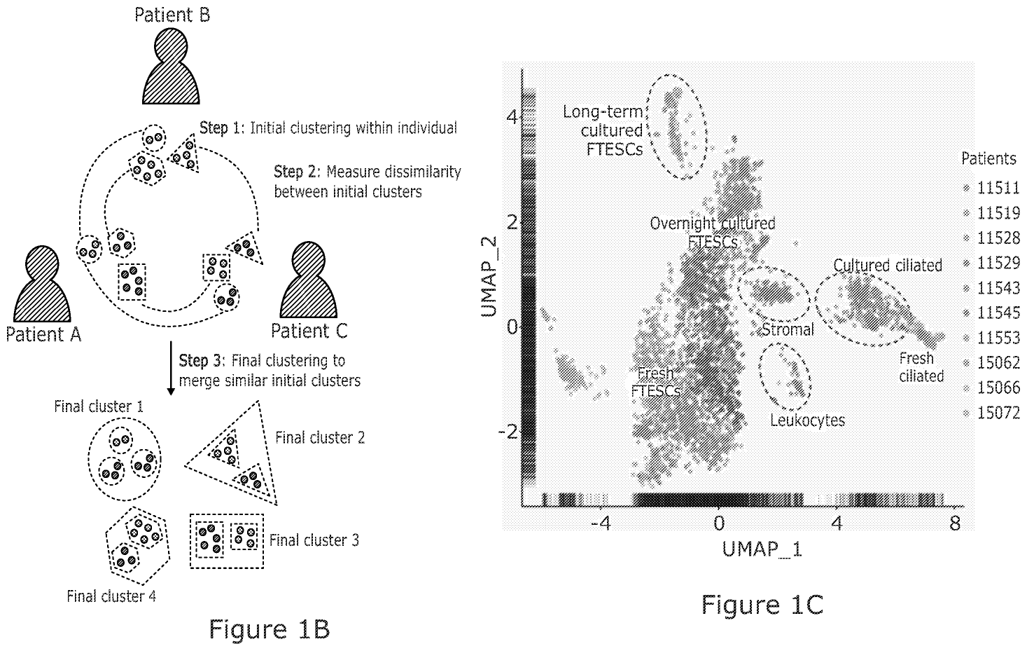

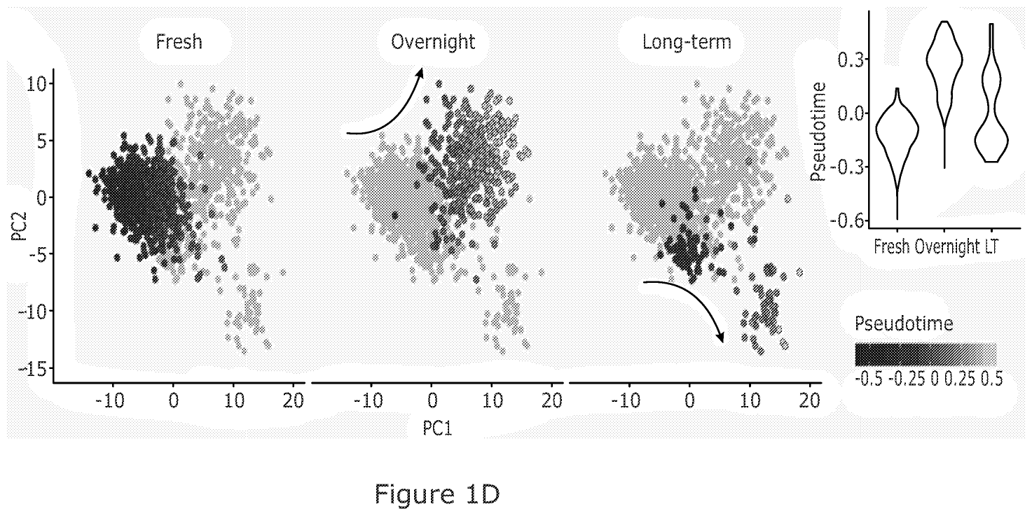

FIG. 1. Landscape of single-cell transcriptome in fallopian tube epithelium.
(A) Diagram shows the single-cell RNA sequencing and analysis workflow.
(B) Diagram of Clincluster shows three steps that help it overcome the confounded batch effects and interpatient variability (see Methods).
(C) Uniform manifold approximation and projection (UMAP) plot profiles 3,800 single-cell transcriptome from fallopian tubes. The cells are colored by their patient sources and annotated with cluster names.
(D) Principal component (PC) plot shows the cells under different condition (fresh, overnight-cultured or long-term cultured) colored by the value of imputed pseudotime values. The violin plot in the smaller panel on the right top shows the pseudotime distribution for each condition. LT, long-term cultured. Pseudotime analysis of secretory cells suggested a more deviated transcriptome after overnight culturing compared to the long-time cultured group. We compared primary secretory cells across three conditions, namely fresh dissociated, overnight cultured and long-time cultured (2-day or 6-day). The cells are plotted based on principal components 1 and 2 (PCI & PC2) and colored by their pseudotime values. Overnight cultured cells have higher pseudotime values than long-time cultured cells

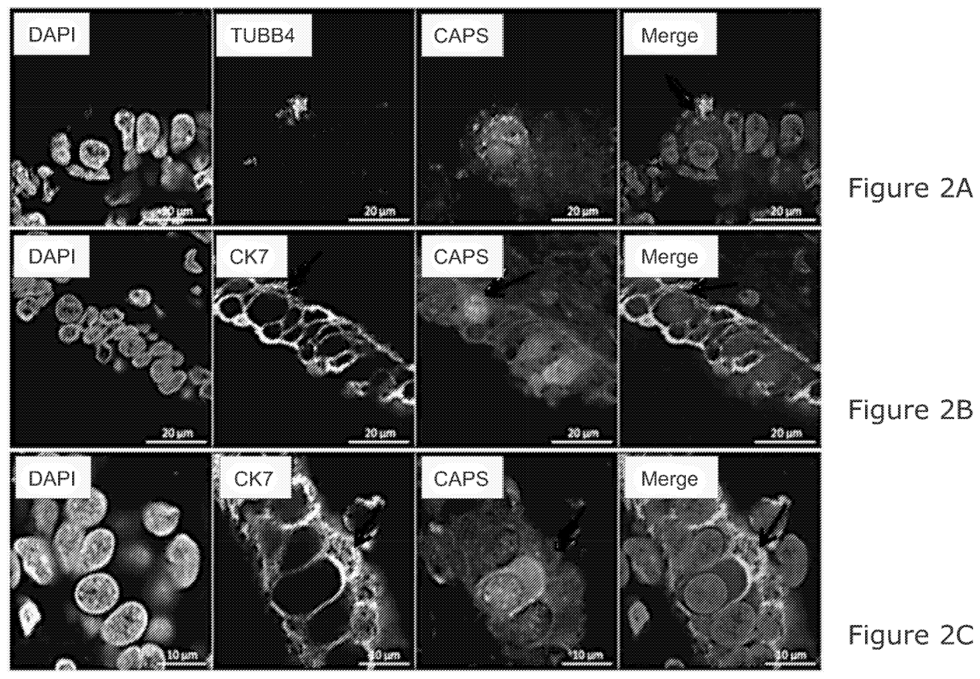

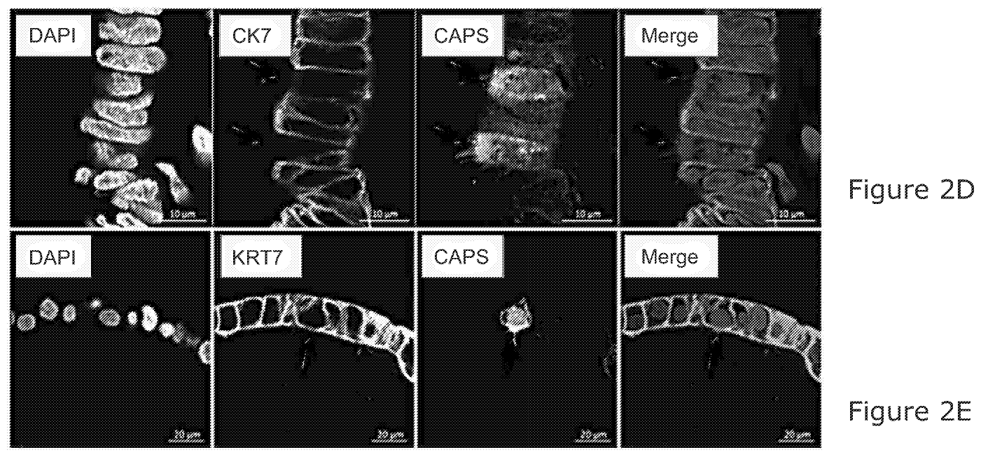

FIG. 2. The immunofluorescent staining validated the existence of the KRT7/CK7 positive and CAPS positive “intermediate” cell type in human fallopian tube epithelium.
(A) The CAPS positive cell (arrow) is also positive for TUBB4, a ciliated marker, validating that CAPS is a ciliated marker.
(B-C) The intermediate cells (arrows) are both KRT7 and CAPS positive, while another CAPS+ ciliated cell in (B) is KRT7 negative. CK7, cytokeratin 7 (KRT7).
(D) The KRT7 positive secretory cells are CAPS negative, while the KRT7 negative ciliated cells (arrow) are CAPS positive.
(E) IF staining in human FTE organoid shows one KRT7+CAPS+ intermediate cell (arrow).

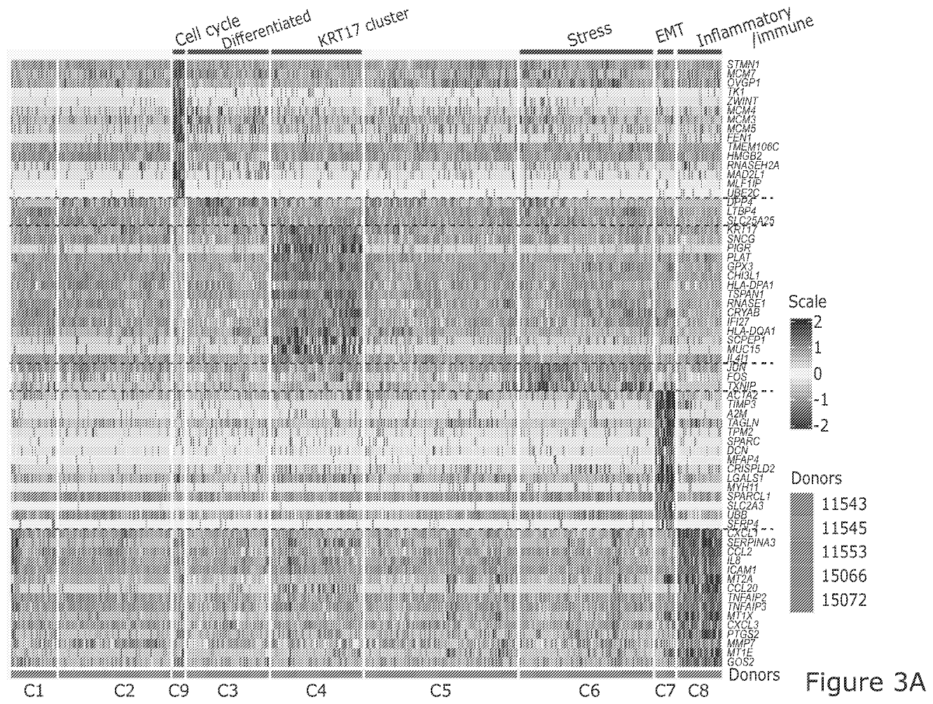

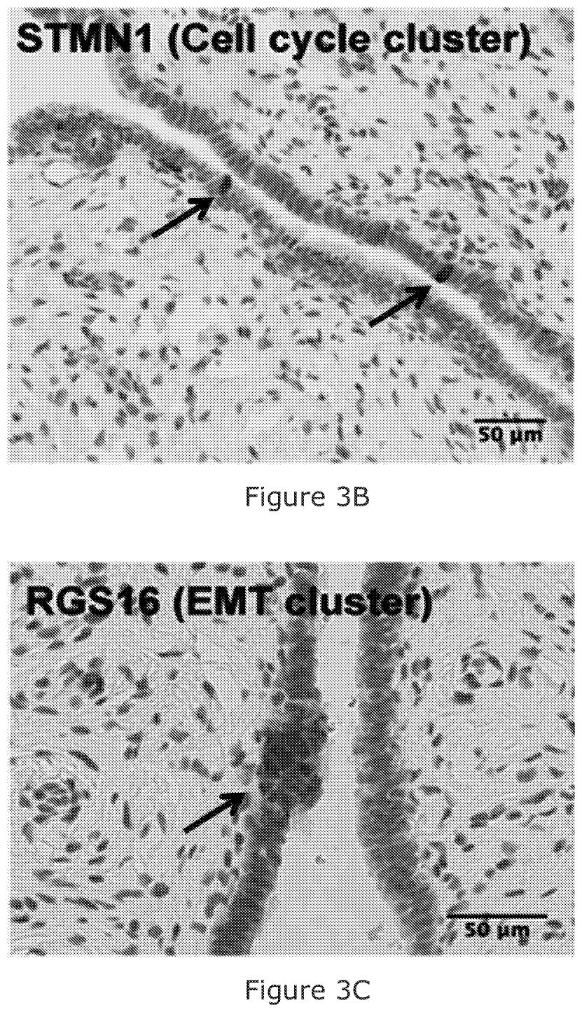

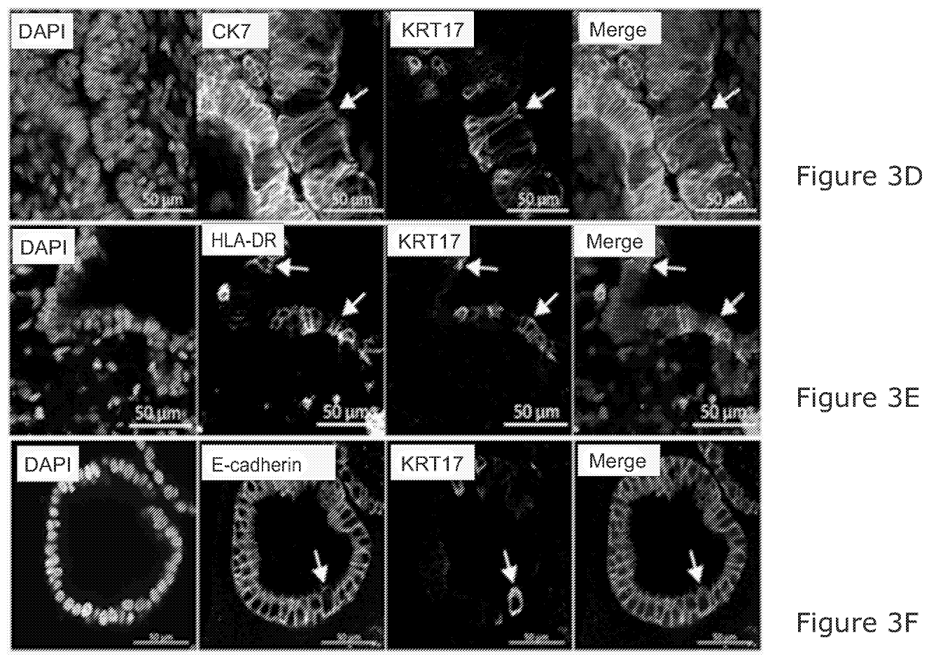

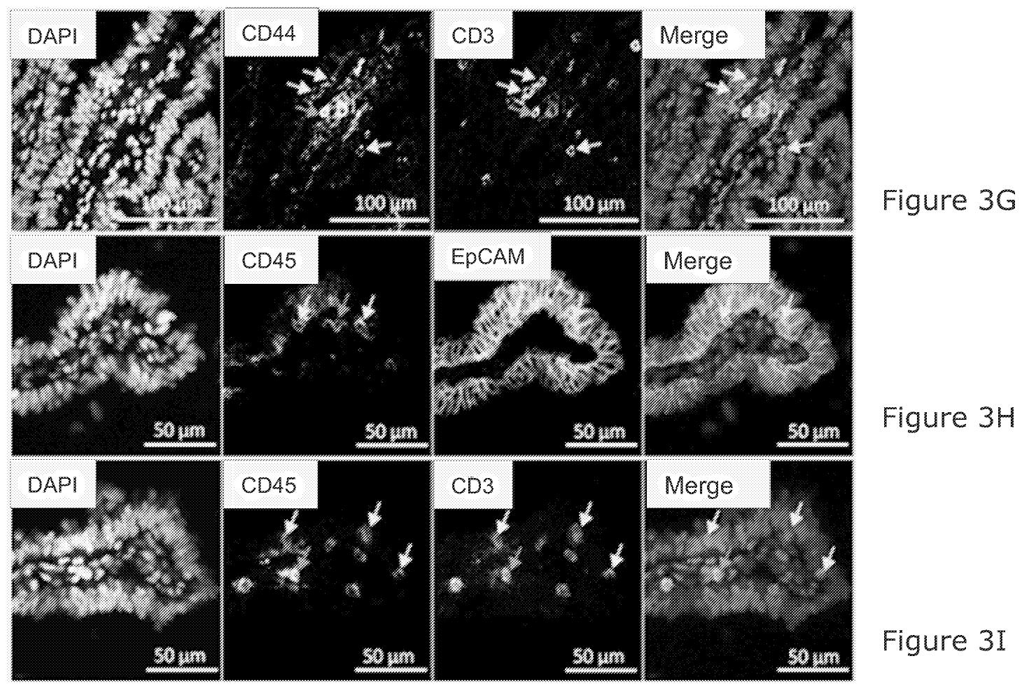

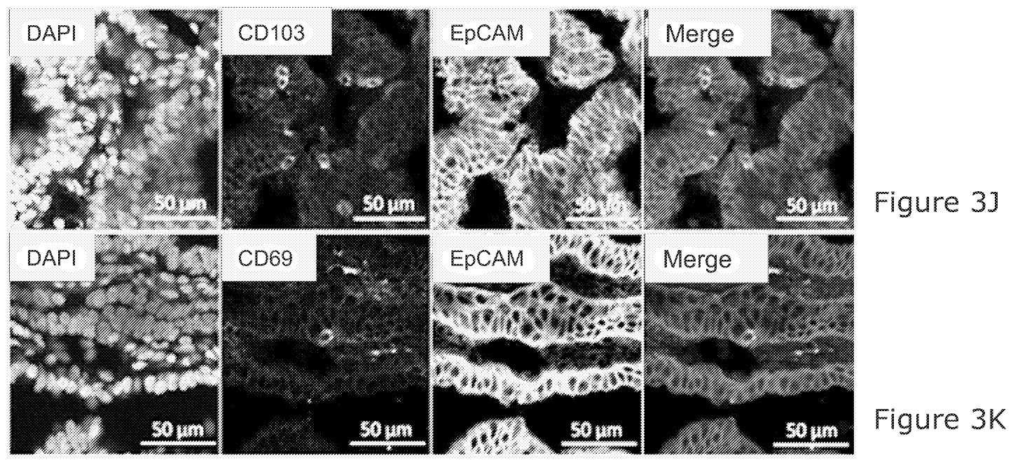

FIG. 3. Subtyping fallopian tube secretory cells.
(A) Heatmap profiling the scaled expression of top marker genes of each cluster in fresh secretory cells.
(B) IHC staining shows the existence and low proportion of Cell Cycle Cluster by its marker STMN1, in human FTE.
(C) IHC staining shows the validation of the marker of ECM Cluster, RGS16, in human FTE.
(D-E) IF staining of KRT17, secretory marker KRT7 (CK7) and HLA-DR in human FTE shows that KRT17 population is secretory cells (D) and has high expression of HLA-DR, an MHC II protein (E).
(F) IF staining of KRT17 and epithelial marker E-cadherin in organoid from human FTE shows that this is a stable population that exists in vivo model.
(G-I) The immunofluorescent staining indicated that the EPCAM+ CD44+ peg cells were positive for lymphocyte markers CD45 and CD3 by double staining of CD44 and CD3 (C), CD45 and EpCAM (D) and CD45 and CD3 (E). The intra-epithelium CD44+CD3+ cells in were also CD45+ and EpCAM+ (yellow arrows). We also observed extra-epithelium CD44+CD3+CD45+EpCAM− cells (red arrows) in the stromal region. It suggests that the basal CD44+ cells are likely positive for lymphocytes markers CD3 and CD45.
(J-K) Immunofluorescent staining suggests that the basal cells are positive for EPCAM and two memory T cell markers, CD103 (J) and CD69 (K).

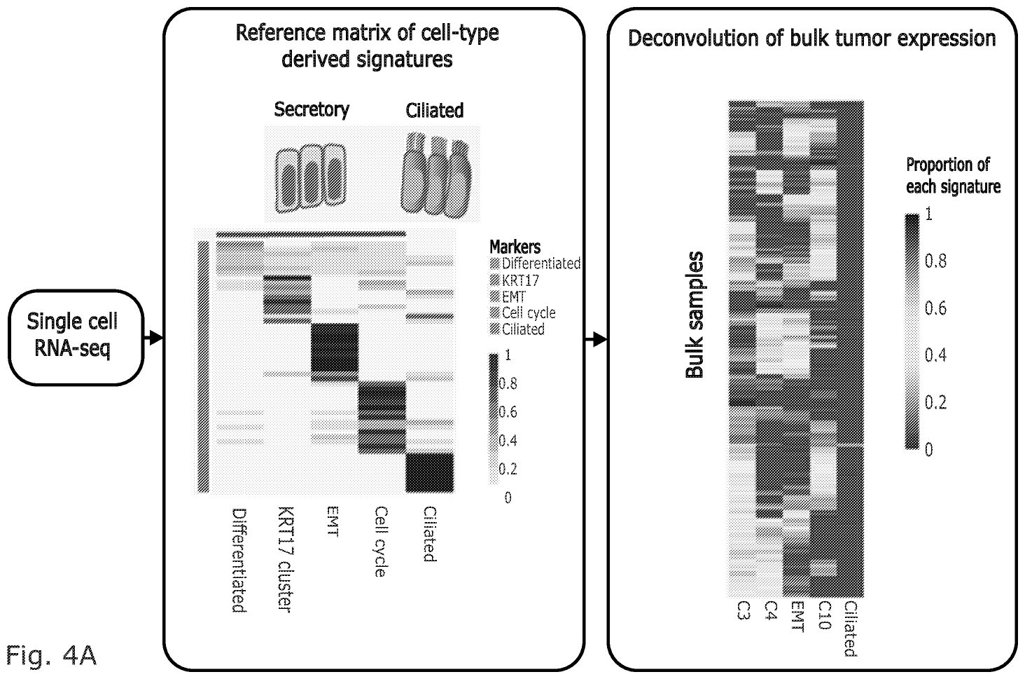

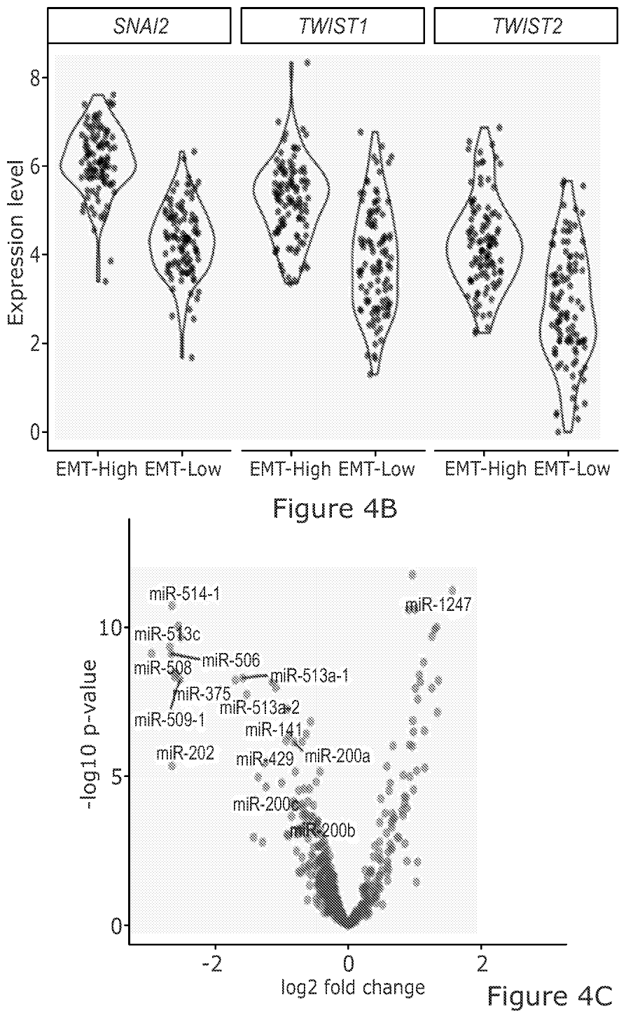

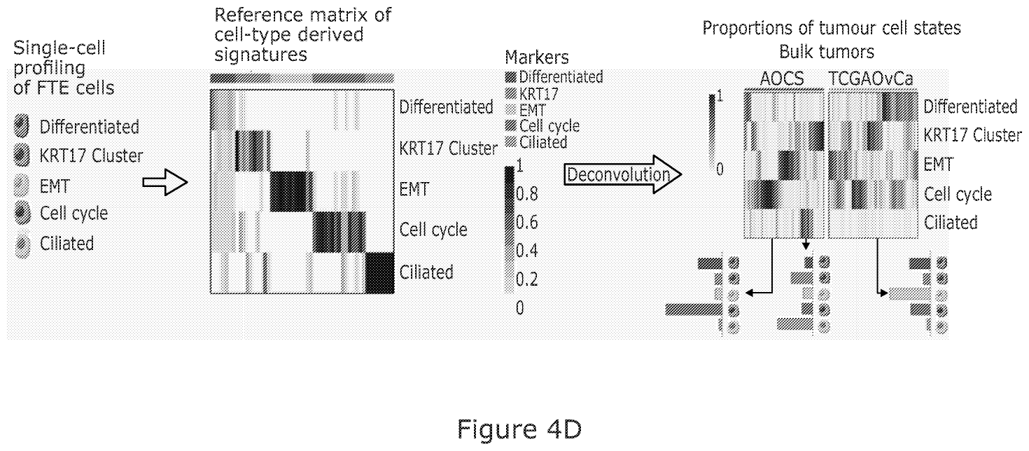

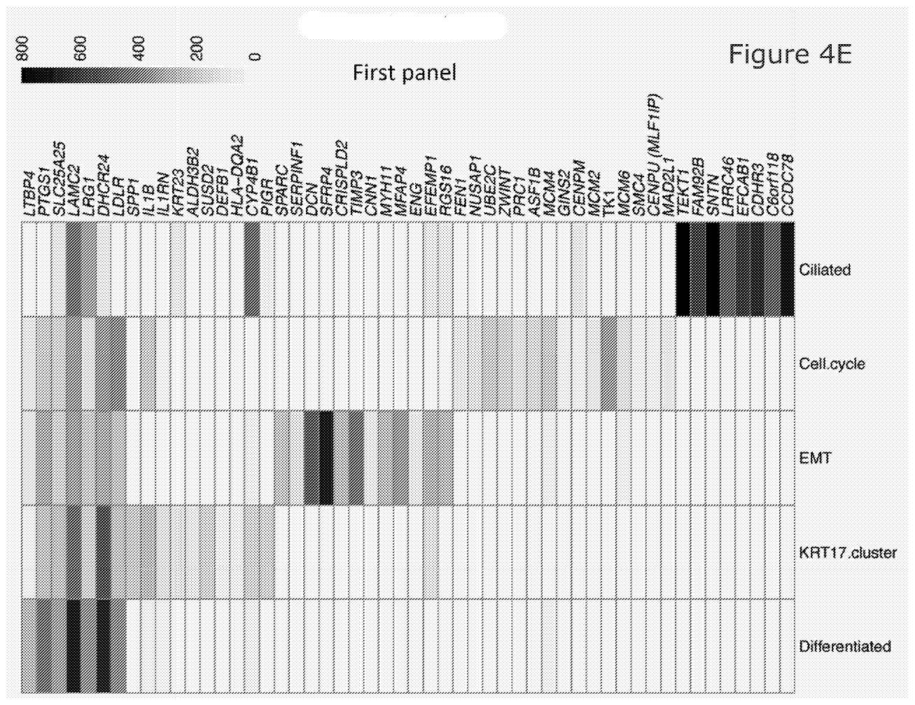

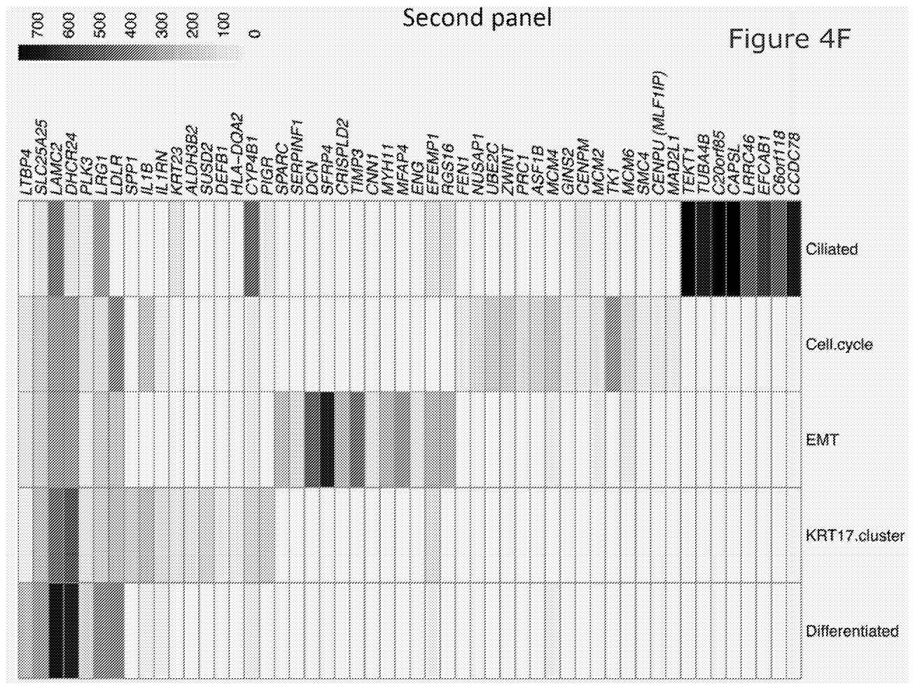

FIG. 4. Deconvolution of bulk expression matrix of HGSOC by FTE cell subtypes revealed a prognostic signature.
(A)Diagram shows the 5-signature panel calculated based on FTE scRNA-seq data by BSEQ-sc. The columns in the heatmap (bottom panel) correspond to five cell subtypes in FTE (top panel). The heatmap stands for the strength of 53 marker genes (rows) in five signatures (columns). A heatmap showing deconvolution results of 308 tumors from the TCGA Ovarian Carcinoma study by Cibersort. The color of cell denotes the proportion (0-1) of five signatures (columns) across tumor samples (rows).
(B) The violin plots showing that expression levels of three key EMT drivers, Twist (TWIST1 and TWIST2) and Snail (SNAI2), are significantly increased in EMT-high tumors (log-FC>1.8, FDR<2e-14).
(C) Volcano plot showing that miRNAs are differentially expressed between EMT-high and -low tumors, including the miRNA-200 family (miR-200a, miR- 200b, miR-200c, miR-141, miR-429), which are the suppressors of EMT. The green and blue dots are significantly differentially expressed miRNAs (log-FC >0.5, FDR<0.05). The blue dots are the ones with text labels next to them.
(D) Shows a diagram of the same 5-signature panel as (A) in a different format.
(E) Shows the same 5-signature panel and heat map as (A) with genes listed according to a first panel of genes,
(F) Shows the same 5-signature panel and heat map with a second panel of genes listed,

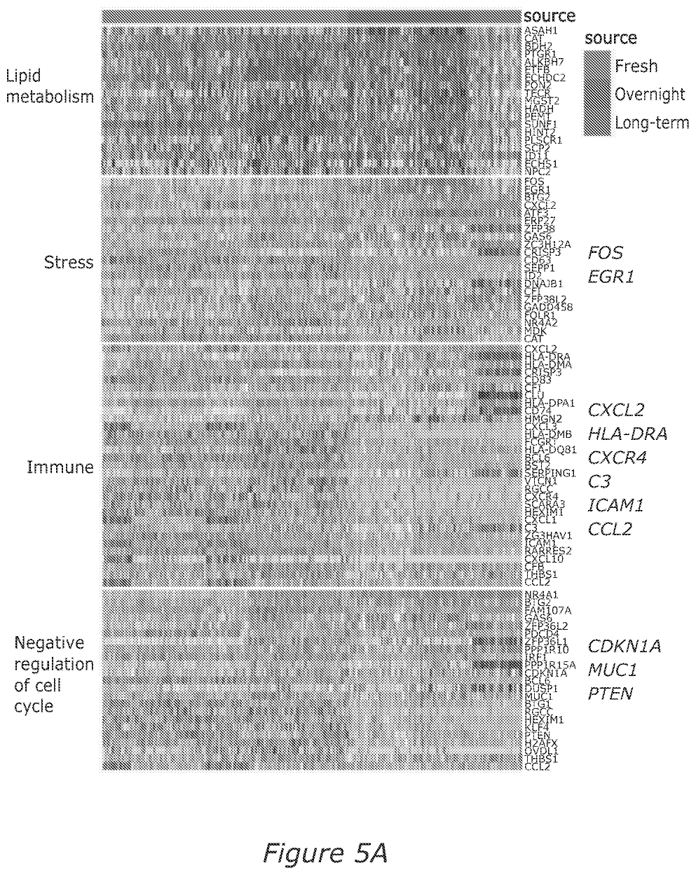

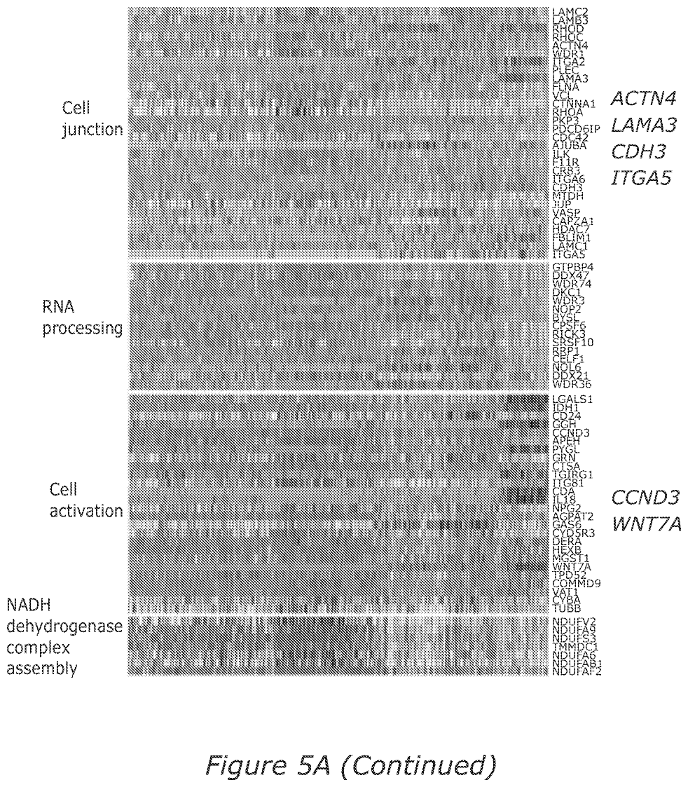

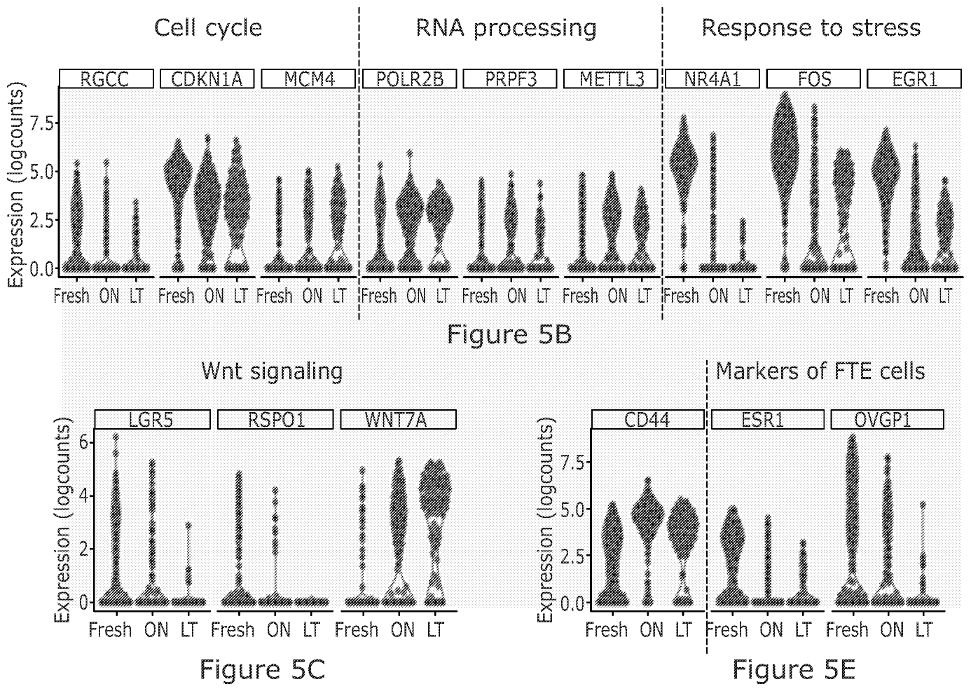

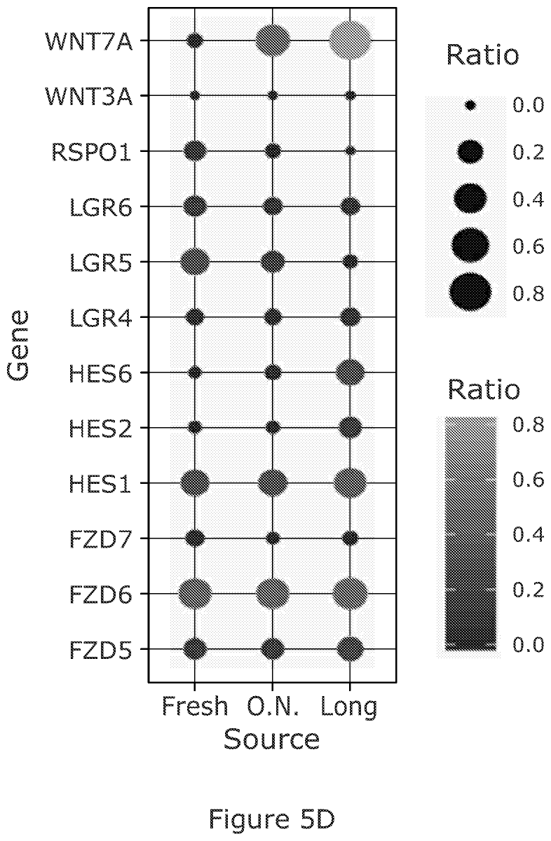

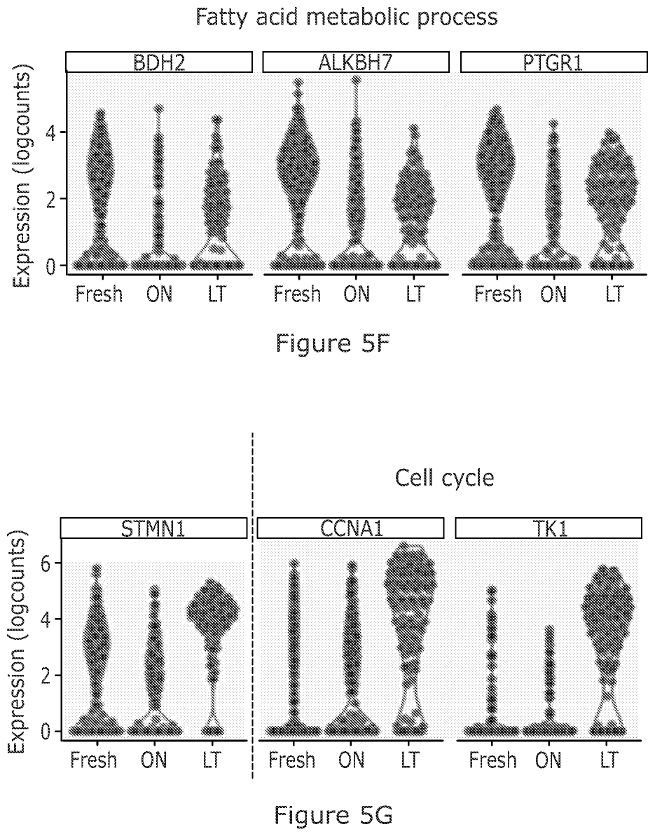

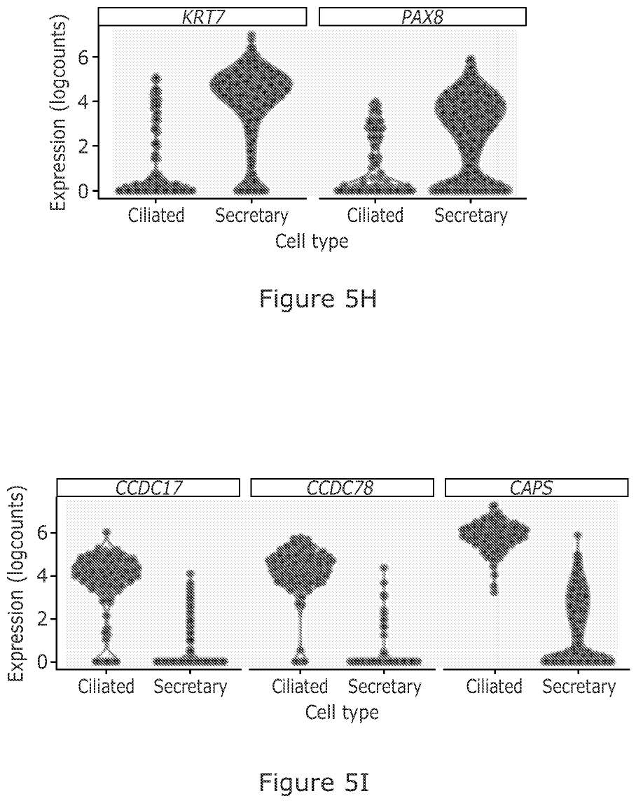

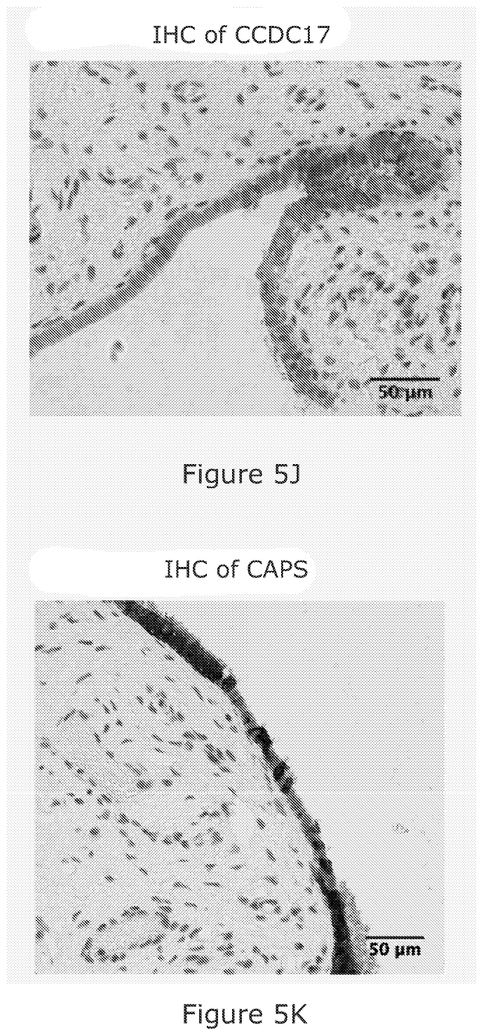

FIG. 5. Landscape of single-cell transcriptome in fallopian tube epithelium and quality control.
(A)Heatmap shows the differentially expressed genes between fresh, overnight-cultured and LT-cultured groups that are enriched in the annotated gene ontology and pathway.
(B) Violin plots show that represented genes in three pathways that are differentially expressed between fresh and overnight-cultured groups (FDR <0.05).
(C) Violin plots show the expression of represented genes (LGR5, RSPO1 and WNT7A) in the Wnt signaling that is disturbed by the culturing condition (FDR<0.05).
(D) Dot plots show that culture condition changes the proportion of cells that express genes related to Wnt pathway, including LGR5, RSPO1, WNT7A and HES6.
(E) Violin plots show the disturbed expression of three genes (CD44, ESR1 and OVGP1) after overnight culturing.
(F) Violin plots show that genes that are enriched in fatty acid processing are transiently switched off after overnight culturing.
(G)Violin plots show that genes (STMN1, CCNA1 and TK1) that are enriched in cell cycle are significantly upregulated and expressed in most of cells after LT culturing.
(H) Violin plots show the expression of marker genes of secretory cells in fresh cells (KRT7 and PAX8).
(I) Violin plots show the expression of marker genes of ciliated cells in fresh cells (CCDC17, CCDC78 and CAPS).
(J) IHC staining of ciliated cell marker CCDC17
(K) IHC staining of ciliated cell marker CAPS

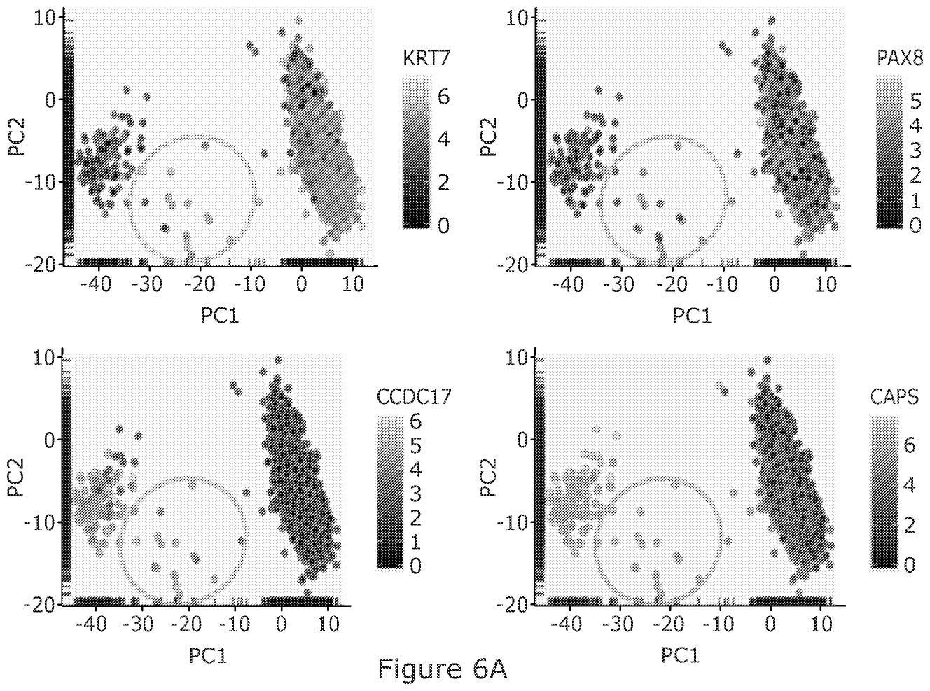

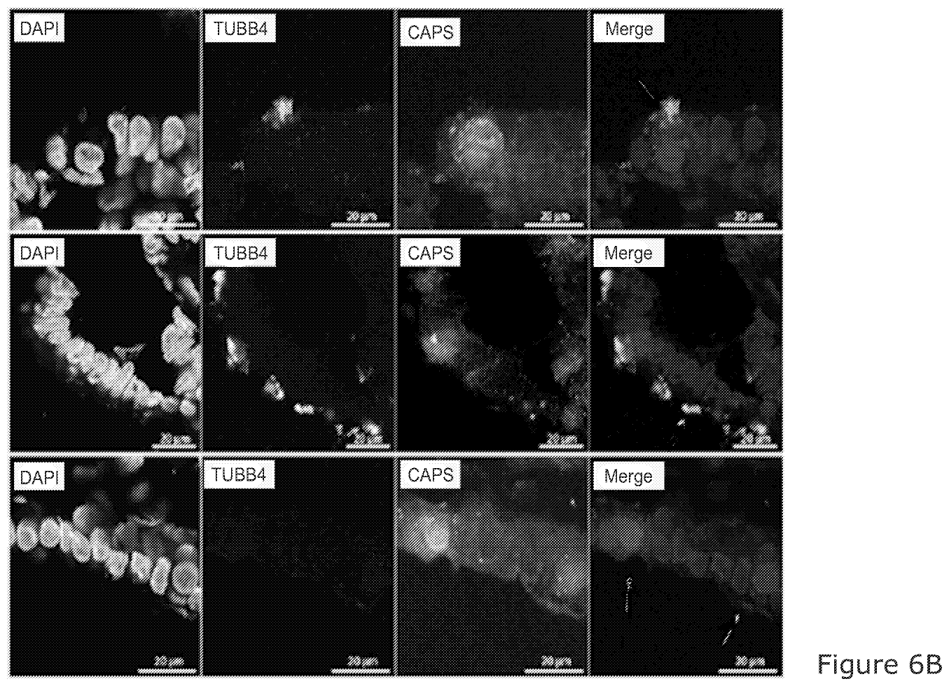

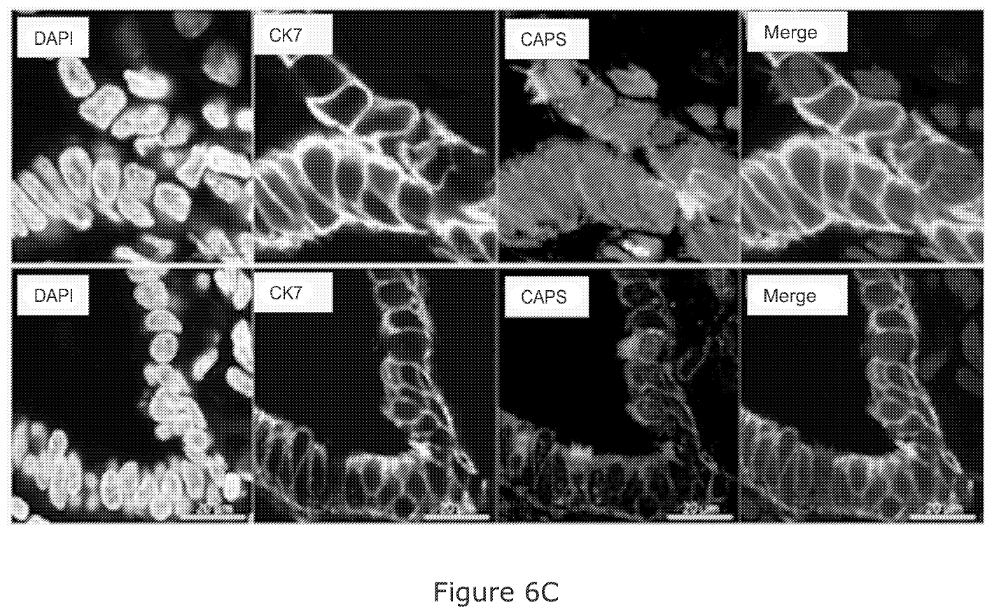

FIG. 6. Intermediate cell subtype.
(A)PCA plots show that the intermediate cell population (grey circle) has the expression of secretory markers (KRT7 and PAX8) and ciliated markers (CCDC17 and CAPS).
(B) IF staining of both TUBB4 and CAPs shows that TUBB4 positive ciliated cells are also CAPS positive. It demonstrated that CAPS is a marker of ciliated cells.
(C) IF staining of CK7 (KRT7) and CAPS shows that the majority of secretory cells are CK7 positive and CAPS negative, while the intermediate cells are double positive.
(D)PCA plots show that the intermediate cell population (grey circle) has the expression of secretory markers (KRT7 and PAX8) and ciliated markers (CCDC17 and CAPS).
(E) IF staining of both TUBB4 and CAPs shows that TUBB4 positive ciliated cells are also CAPS positive. It demonstrated that CAPS is a marker of ciliated cells.
(F) IF staining of CK7 (KRT7) and CAPS shows that the majority of secretory cells are CK7 positive and CAPS negative, while the intermediate cells are double positive.

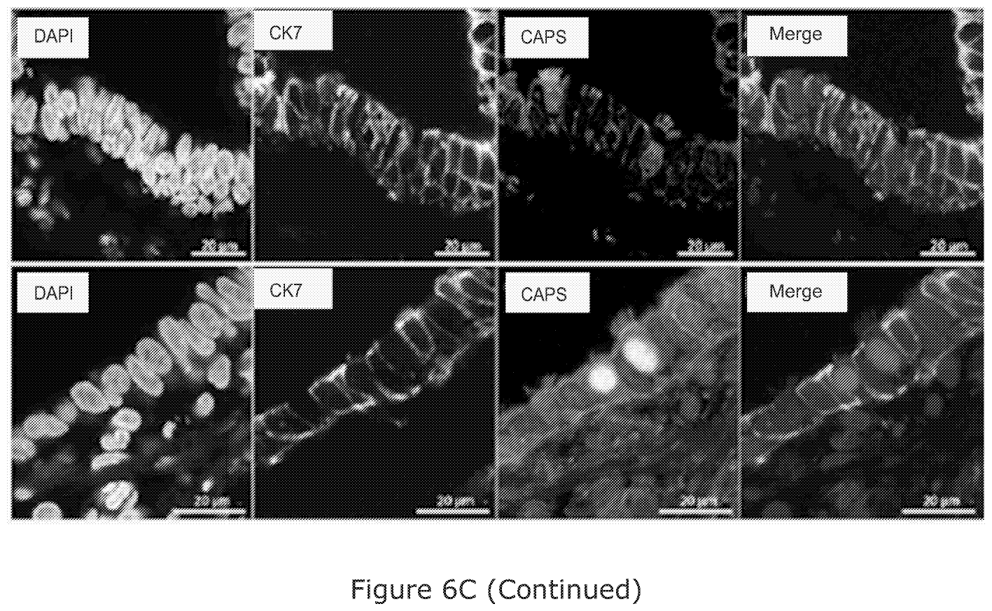

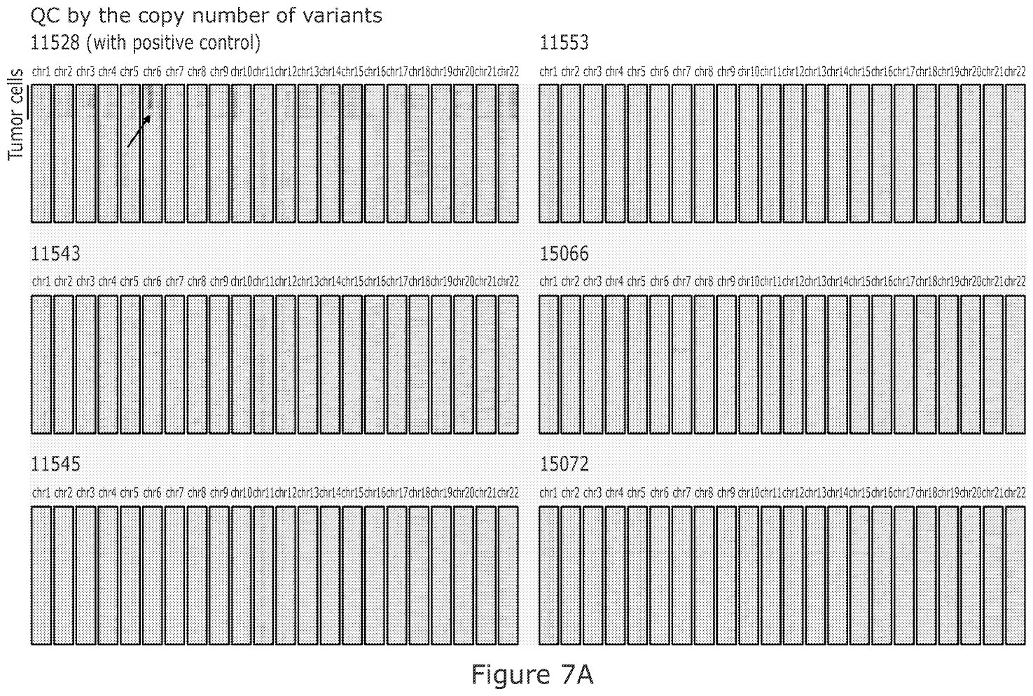

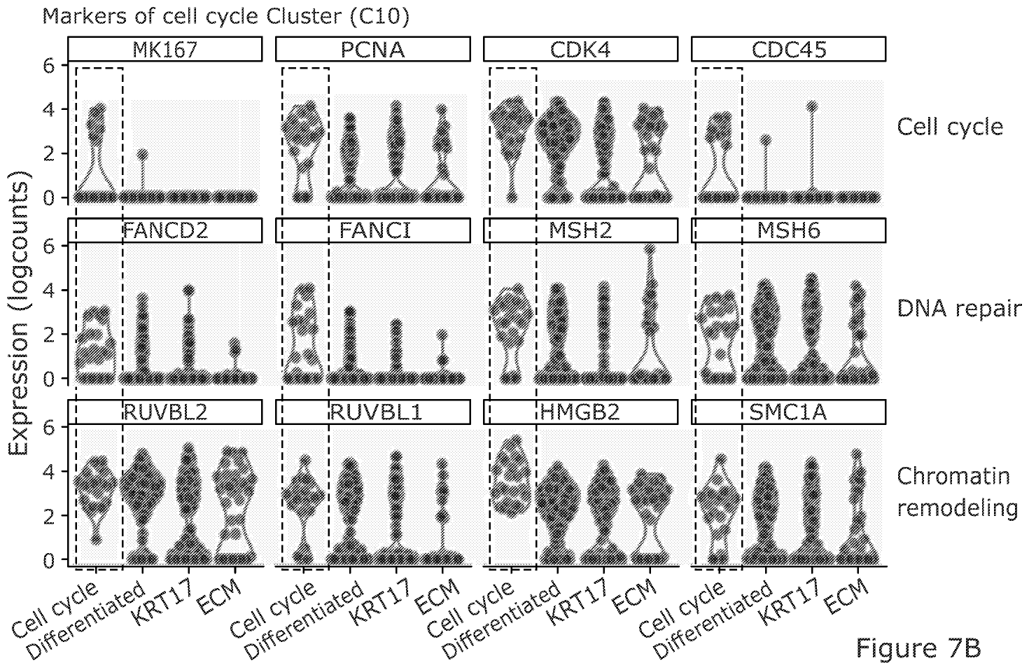

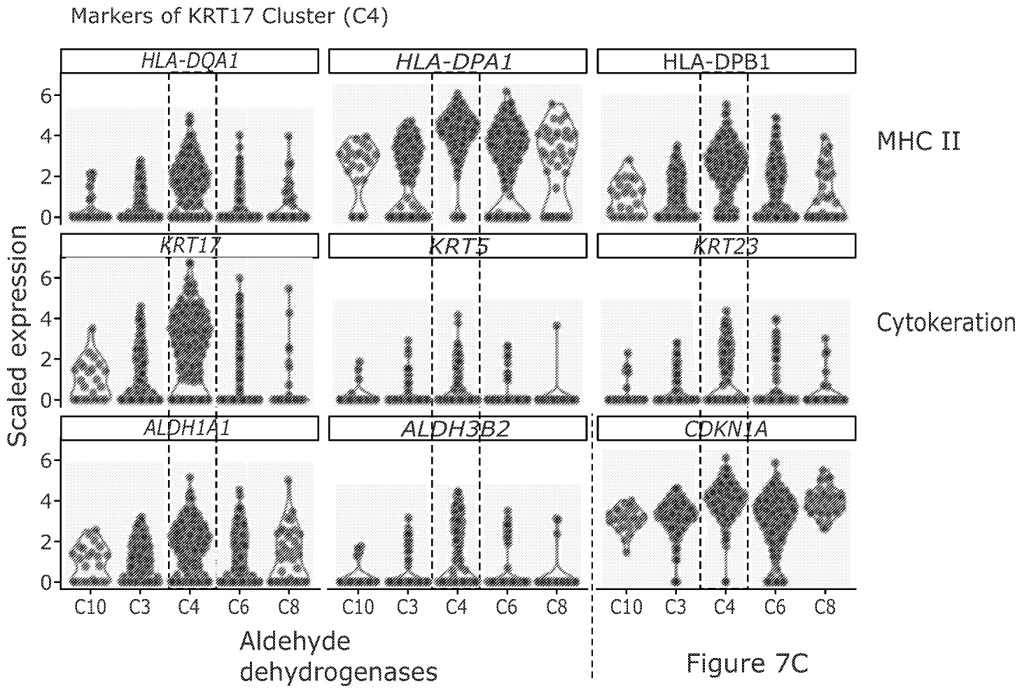

FIG. 7. Novel secretory subtypes and their molecular features.
(A) Quality control of the single FTE cells by the copy number variant referred from expression data.
(B) Violin plots show the upregulation of representative marker genes for Cell cycle cluster (C10), which are enriched in Cell cycle, DNA repair and Chromatin remodeling pathways.
(C) Violin plots show the upregulation of representative marker genes for KRT17 cluster (C4), which involve MHC II, cytokeratin, aldehyde dehydrogenases and p21 (CDKN1A).

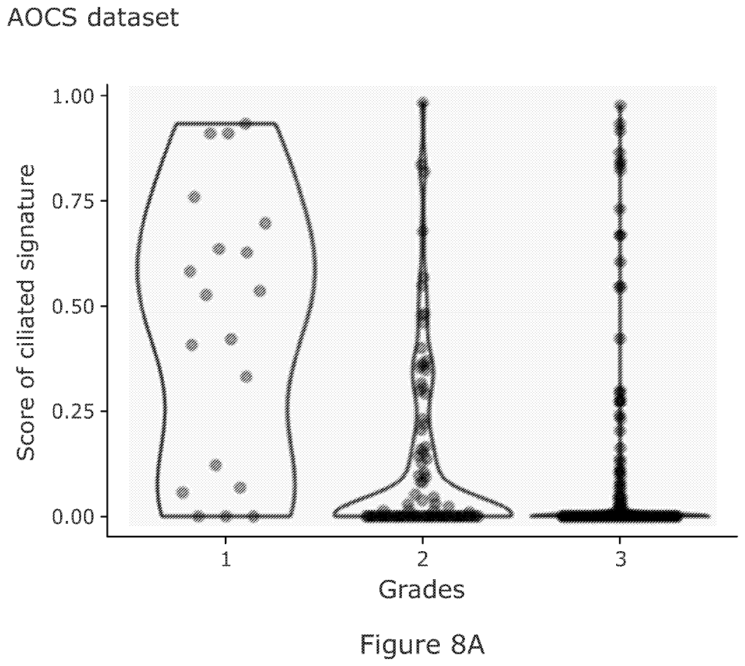

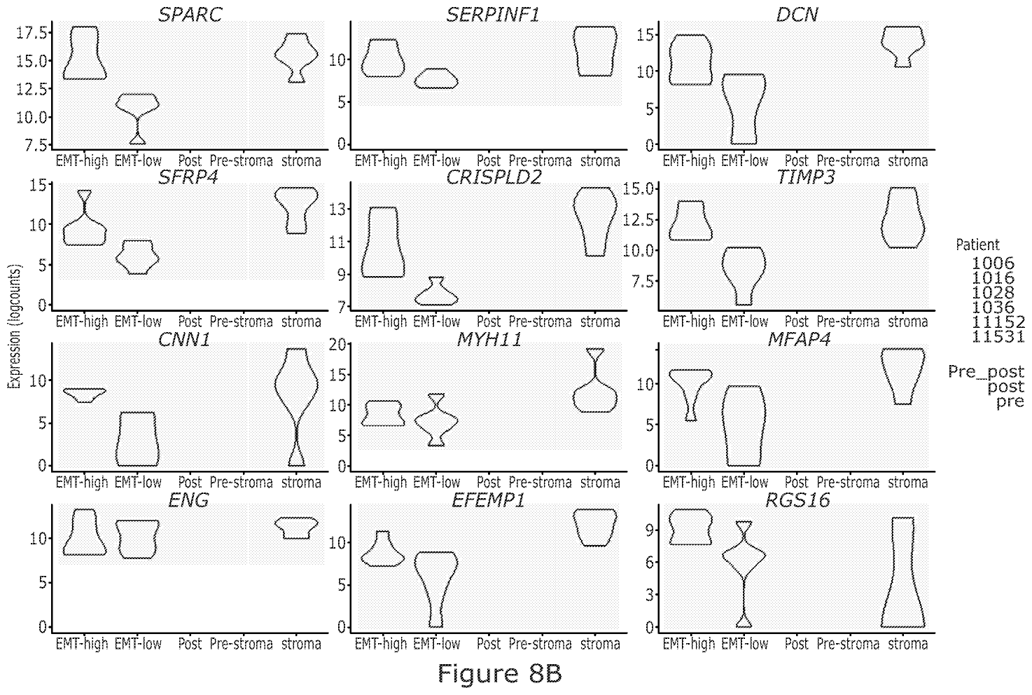

FIG. 8. Deconvolution of bulk expression data of HGSOC.
(A) Violin plot shows that the ciliated signature was enriched in the Grade 1 tumors compared to Grade 2-3 tumors in the AOCS (Australian Ovarian Cancer Study) dataset.
(B) Violin plots show that 6 out of 12 EMT markers are negatively expressed or downregulated in LCM stroma samples compared to LCM tumour samples.

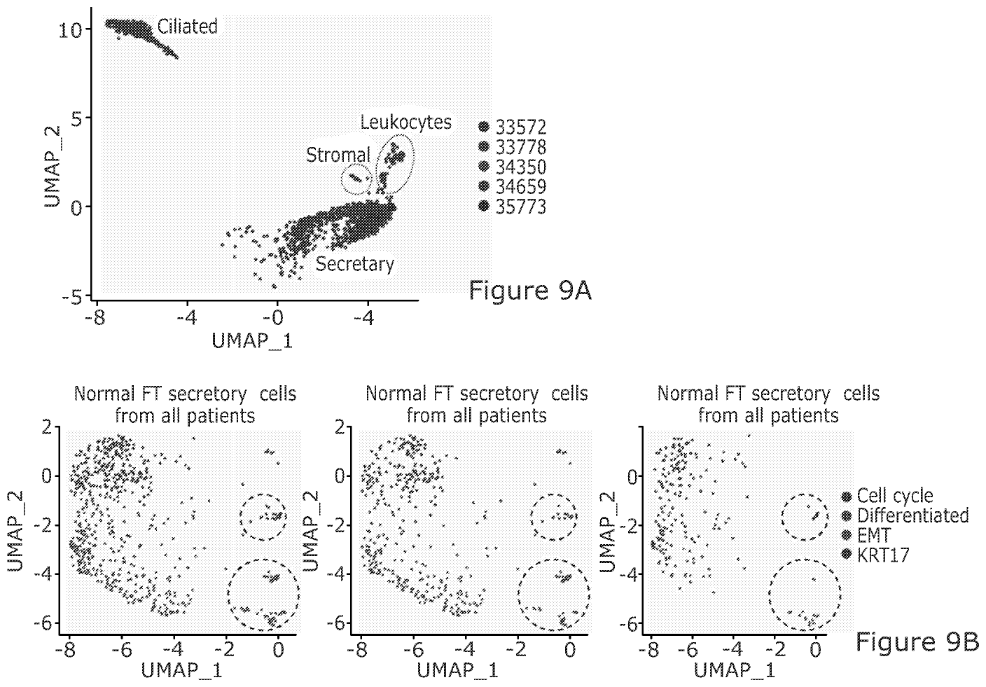

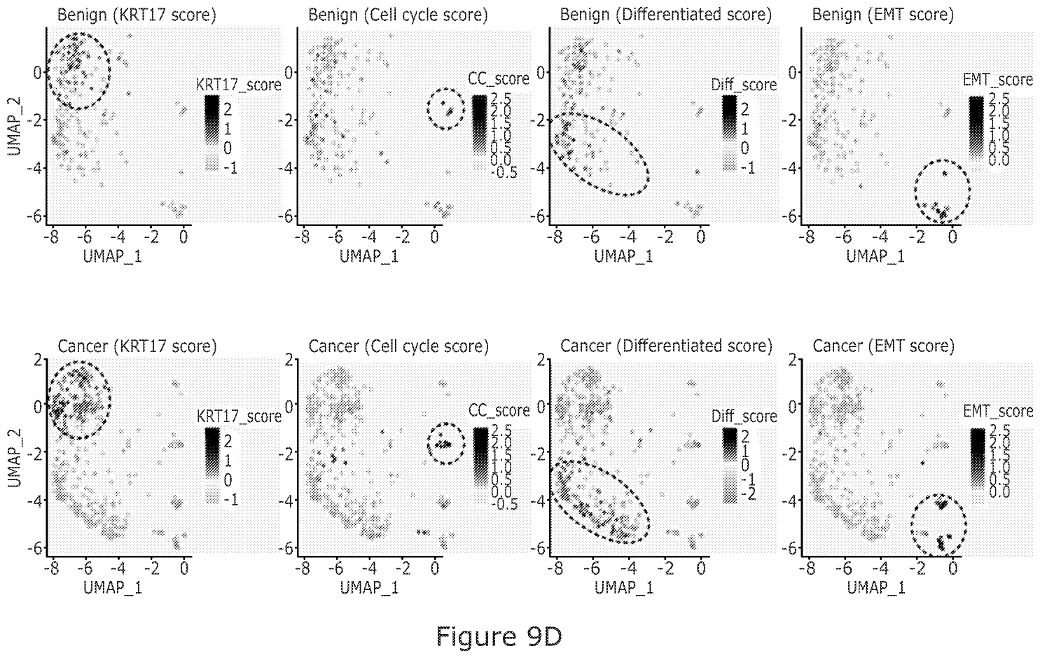

FIG. 9. Validation of the secretory subtypes in the FTE of benign donors by using scRNA-seq.
(A) UMAP shows the populations in the FT samples from 5 benign patients. Each dot is a cell colored by its donor.
(B) UMAP plots show the populations in the FT samples from cancer patients (n =5) and benign patients (n=5). The left, middle and right subpanels contain the cells from all 10 patients, 5 cancer patients and 5 benign patients respectively.
(C) UMAP plot shows the populations in the FT samples from 5 benign patients. Each dot represents a secretory cell from a benign patient. The dots are colored by their donors as shown in the legend.
(D) Scatter plots show the transcriptomic characteristics of each subtype in benign and cancer patients. Cells (dots) are colored by the score of each transcriptomic signature (subtitle). The score of a transcriptomic signature was computed by the scaled and centered sum of expression levels of the marker genes in each transcriptomic signature. The scores correspond to the expression of marker genes of each cluster. The transcriptomic signatures are listed in Table S7.

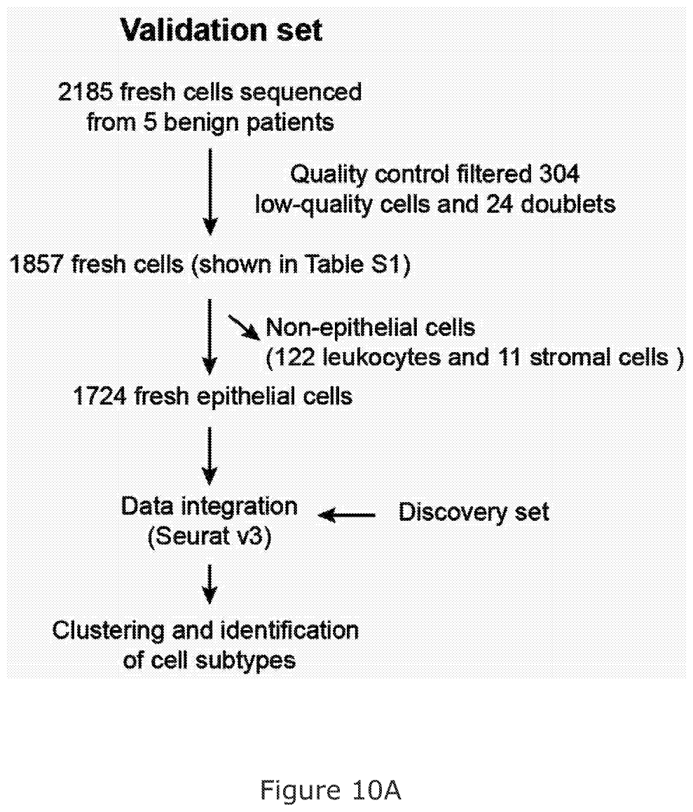

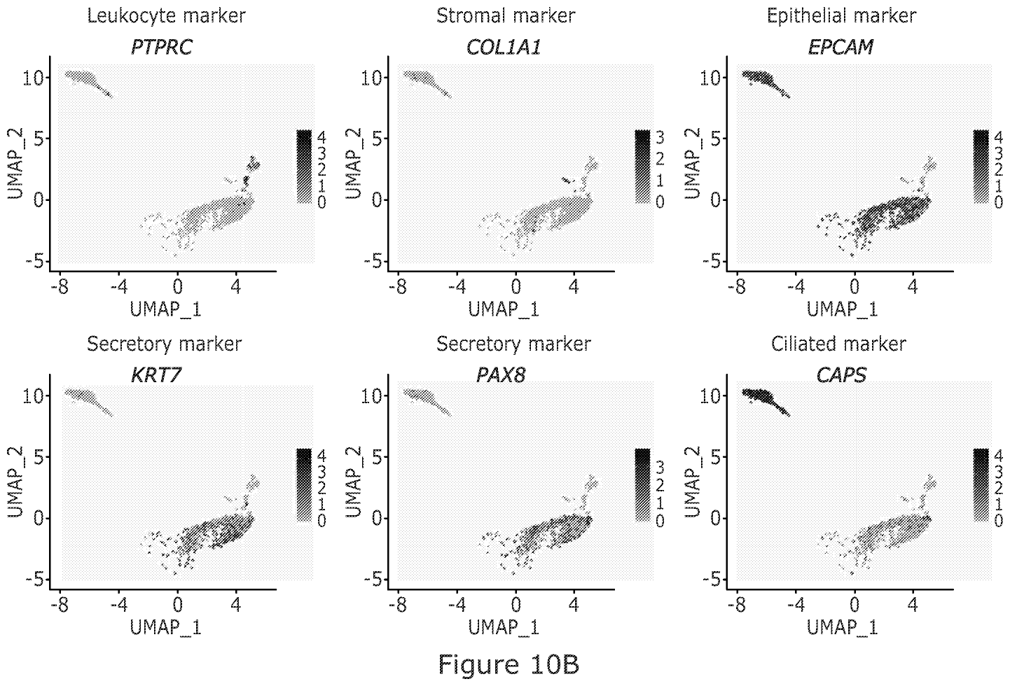

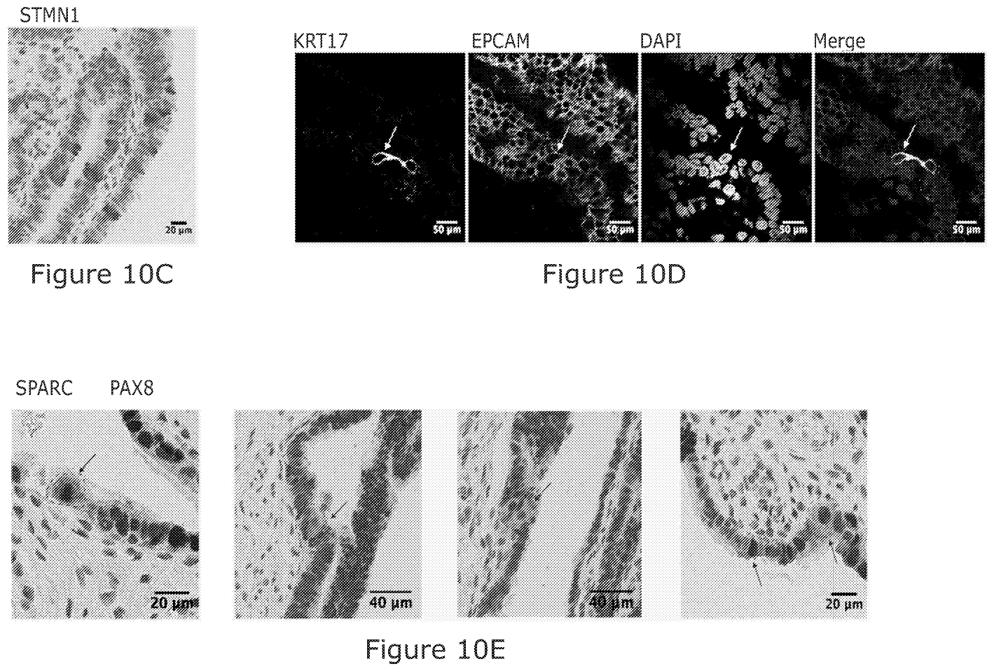

FIG. 10, related to FIG. 3. Validation of the secretory subtypes in the FTE of benign donors by using scRNA-seq.
(A) Flowchart shows the processing of the validation set, in which we profiled 2185 single-cell transcriptomes from five benign patients. After the initial filtering, 1875 cells were left as shown in FIG. 3A. By using the data integration, we removed the batch effects between the discovery set and the validation set and then merged the two datasets. We next clustered the merged datasets to identify the four secretory subtypes in the FTE secretory cells from benign patients.
(B) Scatter plots show the expression of marker genes in the FT cells from five benign patients. The x- and y-axes represent the first two components of the UMAP analysis. Each dot (cell) is colored by the expression level of the marker gene (subtitle). The result shows the CD45+ leukocytes, COL1A1+ stromal cells, KRT7+ PAX8+ EPCAM+ secretory cells and CAPS+ EPCAM+ ciliated cells.
(C) IHC shows the STMN1 positive cell cycle subtype in the FTE of a benign patient.
(D) IF staining shows a KRT17 and EPCAM double positive cell (KRT17 subtype) in the FTE of a benign patient.
(E) IHC images show the SPARC and PAX8 double positive cells in the FTE in the FTE of multiple benign patients (arrows and dashed circles).

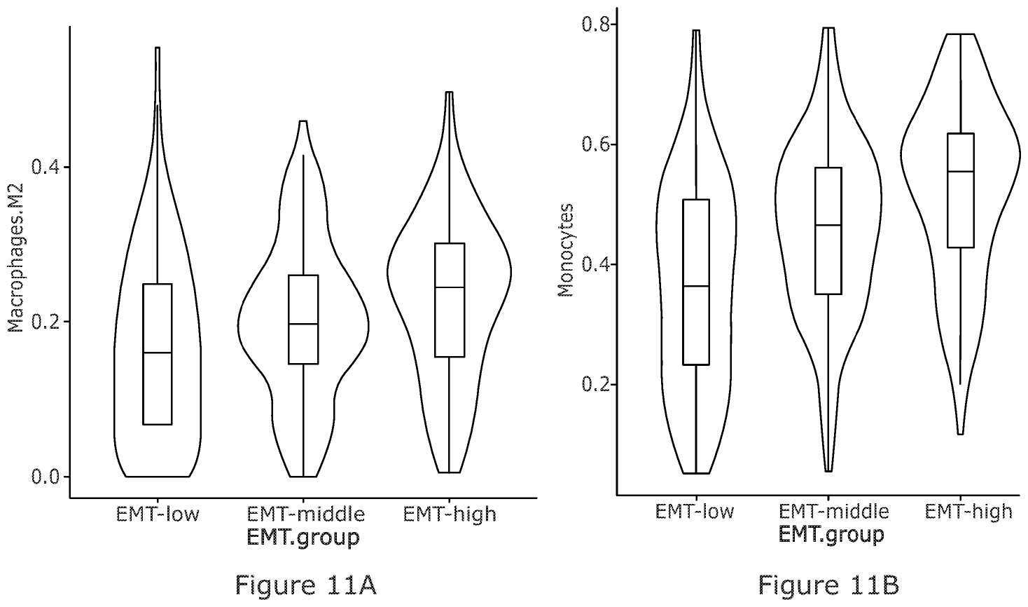

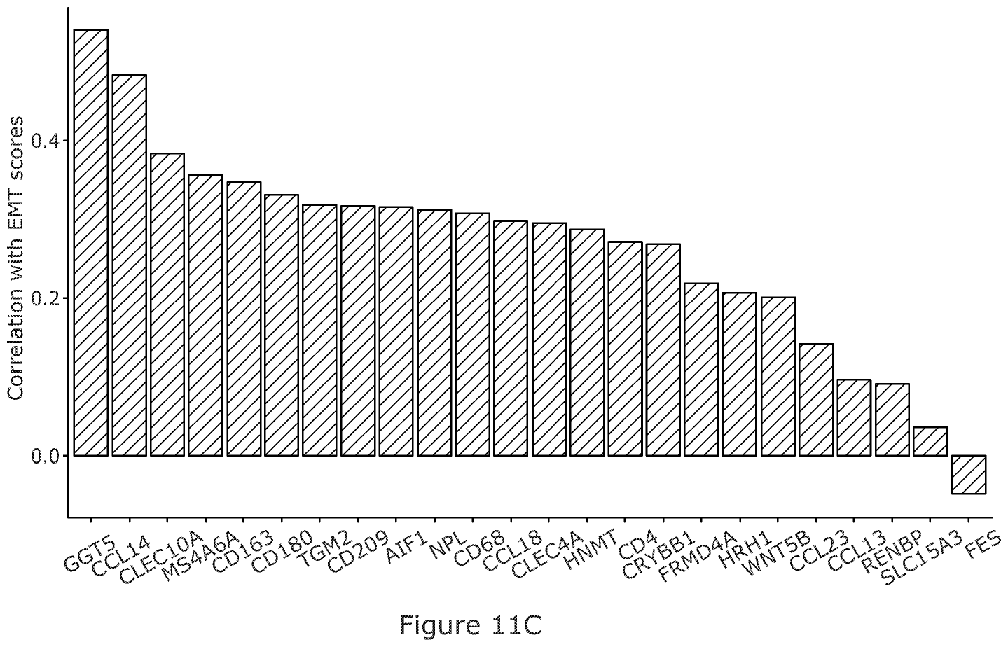

FIG. 11. A—The EMT-high tumours have significantly higher proportions of macrophages M2 compared to EMT-low tumours. p-value=4.093e-05 by one-sided Welch t-test. The y-axis shows the proportion of Macrophages M2. Each dot is a tumour sample from TCGA. B—The EMT-high tumours have significantly higher proportions of monocytes compared to EMT-low tumours. p-value=8.021e-12 by one-sided Welch t-test. The y-axis shows the proportion of Monocytes. Each dot is a tumour sample from TCGA. C—The EMT scores and the expression levels of most macrophage markers are positively correlated. The x-axis is the markers of macrophages M2. The y-axis is the Person correlation coefficient between the EMT scores and the expression levels of marker genes.

### EXAMPLE 1

Introduction

Limited understanding regarding of FTE has hindered further investigation into HGSOC; therefore, cellular subtypes in FTE need to be thoroughly studied at the transcriptomic level. Herein, we profiled the fallopian tube epithelium from patients with HGSOC or endometrium cancer to delineate subtypes in FTE secretory cells and their marker genes. These markers from FTE single cells were then used to stratify HSGOCs and identified a tumor subtype with poor overall survival.

Results

A Cell census of human fallopian tubes in cancer patients We analyzed 3,877 single cells from the fallopian tubes of six ovarian cancer patients and four endometrial cancer patients using Smart-Seq2 technique (Picelli et al., 2014) (FIG. 1A and Table 2). Flowcytometry was used to identify and sort single epithelial cells (EPCAM+, CD45−), leucocytes (EPCAM-, CD45+) and stromal cells (EPCAM−, CD45−) prior to sequencing. To overcome the confounding of batch effects and patient-specific variability in clinical samples, we used differential-expression-based clustering. This clustering approach is based on a functional similarity assumption, in which the differential expression (DE) patterns across batches are similar but distinguishable across cell populations with miscellaneous functions (FIG. 1B). Using this approach, we were able to differentiate between epithelial and non-epithelial cells (FIGS. 1C).

However, we observed striking effects of the culture conditions on the single cell transcriptomes. Most notably, overnight culturing, induced profound differential expression changes in pathways related to cell cycle (e.g. RGCC, p21 and MCM4), RNA processing (e.g. POLR2B, PRPF3 and METTL3) and stress response (e.g. NR4A1, FOS and EGR1) (FIGS. 5A and B). In addition, the Wnt signaling pathway was also significantly affected, where LGR5 and RSPO1 were downregulated and WNT7A was upregulated after culturing (FIGS. 5C and D). Importantly, overnight culture induced the expression of genes that are known to be rarely expressed in FTE cells such as CD44 (logₑ fold-change [log-FC]=3.8) (Paik et al., 2012) and reduced the expression of key markers of secretory cells such as Estrogen Receptor alpha (ESR1) and Oviductal Glycoprotein 1 (OVGP1) (FIG. 5E) (Cerny et al., 2016; Wu et al., 2016). Cilium organization was also downregulated in the overnight-cultured ciliated cells. Moreover, Pseudotime analysis (Campbell and Yau, 2018) across three conditions from the same patients revealed that the transcriptomes of fresh cells were more similar to the long-term (LT) cultured cells compared to the overnight group of cells (FIG. 1D). For instance, fatty acid metabolic process (e.g. BDH2, ALKBH7 and PTGR1) was provisionally downregulated after overnight culturing and then upregulated in the LT group, while RNA processing pathway was upregulated (FIGS. 5A and 5F). This suggested that including the overnight-cultured cells in subsequent analysis may introduce significant biases that would preclude meaningful conclusions. Similarly, although the LT group resembled the fresh group of cells, they also showed a unique split into two subgroups and disturbed expression of Stathmin and cell cycle genes that probably represent an artefact of long-term culture (FIG. 5G). To avoid the substantial effects from preservation methods, we focused our analysis on fresh cells only.

Within the epithelial cells, we identified the two previously established subtypes, secretory and ciliated cells (FIG. 1C). Secretory cells were characterized by the expression of PAX8 and KRT7 (FIG. 5H) as well as a large number of newly identified markers of secretory cells. The ciliated population was represented by high expression of the FOXJ1 and members of the coiled-coil domain containing protein family, such as CCDC17 and CCDC78 (FIGS. 51 and J). This protein family is essential for cilia functioning (Klos Dehring et al., 2013). We also identified a list of previously unrecognized markers of fallopian tube ciliated cells such as the calcium binding protein Calcyphosin (CAPS) that were enriched in the cilium-related pathways (FIGS. 5I, K and 2A) (Wang et al., 2002).

In addition to the two established cell types, we discovered a rare intermediate type that was characterized by the expression of the secretory cell marker KRT7 and high expression of ciliated marker CAPS (FIGS. 2B, C and 6A), whilst other KRT7+ secretory cells were CAPS negative and validated this population in human FTE (FIGS. 2D and 6B). PAX8 was expressed in a subset of this population, possibly because that its moderate expression level caused a higher dropout rate. Additionally, this subtype was enriched in overnight cultured cells and recapitulated in organoids cultured from human FTE (FIG. 2E). However, due to the low proportion of this intermediate population, it is challenging to conduct DE analysis to identify specific markers for this population. This intermediate population might represent an intermediate cell state between secretory and ciliated cells, which accords with the previously assumed transition between these two cell types (Ghosh et al., 2017; Hellner et al., 2016).

Four Novel Secretory Subtypes in FTE

We next attempted to classify secretory cells based on their transcriptomes. To ensure the purity of secretory cells, the cell was only kept for further analysis if it had strong expression of KRT7 and EPCAM and no expression of CCDC17 or PTPRC. In addition, to avoid including contaminating cancer cells, we excluded cells that had detectable copy numbers variants or loss-of-heterogeneity (FIGS. 7A) (Fan et al., 2018). By applying the DE-based clustering on fresh secretory cells, we found that they were clustered into nine clusters (FIGS. 3A). Except for a patient-specific cluster (C8) that was enriched in inflammatory markers, all other clusters contained cells from multiple patients. Three out of nine clusters (C1, C2 and C5) had no particular distinguishing features suggesting that they probably represented a quiescent population of cells. Cluster C6 had evidence of cell stress as shown by high expression of early response genes such as FOS and JUN (Honkaniemi et al., 1992).

Surprisingly, C7 showed high expression of a Regulator of G protein signaling (RGS16) and genes that were enriched in the extracellular matrix (ECM) pathway (false discovery rate [FDR]=1.80E-17), such as TIMP3, SPARC and COL1A (FIGS. 3B and C). We validated the presence of this subtype using IHC (FIGS. 3B). This cell type may be generated by the epithelial-mesenchymal transition (EMT), which can be induced by the chronic exposure to the oxidative stress (Mahalingaiah et al., 2015) and might be related to cancer development (Hanahan and Weinberg, 2011). This cell type is not a contamination from FT mesenchymal cells because it strongly expressed KRT7 and EPCAM (FIGS. 7D).

Cluster C3 had upregulation of genes that are involved in RNA synthesis and transport (e.g. PTBP1, ZNF259 and PRPF38A). It probably represented a transient differentiating cell population. Cluster 4 is characterized by the upregulation of major histocompatibility complex (MHC) Class II genes (e.g. HLA-DQA1, HLA-DPA1 and HLA-DPB1), cytokeratins (KRT17 and KRT23), aldehyde dehydrogenases (e.g. ALDH1A1 and ALDH3B2) and CDKN1A (also called p21) (FIGS. 3A and 7D). KRT17 was reported to be expressed in around 5% of FTE cells (Comer et al., 1998), but the fact that it was enriched in MHC class II expression was unknown. Importantly, this KRT17 positive cluster was validated in human FTE using IF and recapitulated in organoid cultures grown from human fallopian tube epithelial cells suggesting that they represent a robust group of cells with potentially important biological functions (FIGS. 3G-I).

C9 cluster (−1.6% of fresh FTESCs) most probably represented cycling cells because the marker genes of this cluster were enriched in three pathways, namely cell cycle (e.g. MCM2-7, MKI67, TK1 and STMN1), DNA repair (e.g. FANCD2, FANCI and MSH2) and chromatin remodeling (e.g. HMGB2 and SMC1A) (FIGS. 3A, B and 7B). MKI67 (also known as Ki-67) is a well-known marker for proliferation in FTE and other cells (Kuhn et al., 2012). The two Fanconi Anemia genes, FANCD2 and FANCI, can form a heterodimer that is essential for DNA repair and can interact with MCM2-7 (Nalepa and Clapp, 2018). The relatively low percentage of cycling cells is consistent with the age of the patients from whom the cells were obtained.

We also confirmed the CD45+EPCAM+ population that was located as basal cells in FTE by IF staining (FIG. 3H). This population was also positive for CD3, CD44, CD69 and CD103 (FIGS. 3G and I), suggesting that they are likely tissue resident T lymphocytes.

Deconvolution Revealed a Poor-Prognostic Tumor Subtype

We hypothesized that FTE cell subtypes might be correlated with HSGOC tumor types. Based on the four novel secretory subclasses and the ciliated cell type, we firstly computed a reference matrix with cell-type derived transcriptomic signature from five major FTE cellular subtypes (Cell cycle, EMT, Differentiated, KRT17 cluster and ciliated) as previously described (FIG. 4A) (Baron et al., 2016). The resulting signature matrix was then used for the deconvolution analysis (Newman et al., 2015) on bulk ovarian cancer RNA-seq data from The Cancer Genome Atlas (TCGA) and the microarray date from AOCS study (Bell et al., 2011; Tothill et al., 2008) to generate the fractions of five subtypes within each tumor. Whereby, we found a dispersed proportion of composition across tumors for these five cell types (FIG. 4A). Over 75% (233/308) tumors from TCGA were dominated by one cellular subtype (fraction >0.5), while the rest tumors have main components from multiple subtypes. For example, the ciliated tumor subtype was enriched in the Grade 1 tumors compared to Grade 2-3 in the AOCS dataset (p<1e-06, one-sided Wilcox test, FIG. 8A), suggesting that the grade of serous ovarian carcinoma may be related to their ability to differentiate. Most notably, we were able to identify a class of EMT-enriched tumors in multiple data sets. These tumors were enriched in genes that were previously linked to the “mesenchymal” HGSOC subtype. We found that the marker genes (FDR<0.05, log-FC>1) of these tumors were enriched in focal adhesion and PI3K-Akt signaling pathway (FDR<0.0002, by DAVID), which are critical for tumor cell survival (Fresno Vara et al., 2004; McLean et al., 2005). Furthermore, three key EMT genes, TWIST1, TWIST2 and SNAI2 (Ansieau et al., 2008; Kang and Massagué, 2004; J. Yang et al., 2004), were upregulated in the EMT-high tumors (FIG. 4B), suggesting that EMT may be the underlying mechanism of this tumor subtype. To test whether the EMT-high tumor subtype was merely caused by stromal cell impurities in tumor samples, we performed RNA sequencing on 36 laser capture microdissected (LCM) tumors from samples collected from 15 patients and classified tumors based on the deconvolution analysis. We compared the expression of genes in EMT signature between laser capture microdissected tumor and stroma samples. As expected, expression levels of PAX8 and EPCAM were significantly higher in tumor samples compared to stromal samples in which these markers showed almost no expression (FIG. 8B). In contrast, EMT-high markers (SPARC, TIMP3 and MFAP4) were highly expressed in both tumor and stroma confirming that EMT-high tumors truly express these genes. Furthermore, we also verified that the EMT-high tumors were not the by-product of ploidy or copy number aberrations of the involved highly expressed genes.

We next tested whether any of the five tumor subtype scores from the deconvolution analysis correlated with survival. The EMT score was significantly associated with poor overall survival and was independent of the effect of ages, stages and residual diseases (p<0.05, by Cox proportional hazard model). The robustness of the association was confirmed by the permutation test (n=500) leaving out 10% samples each time (empirical p-values=0.012 [TCGA] and 0 [AOCS], permutation test).

SPARC, one of the 12 genes that comprise the EMT signature, was previously described in the mesenchymal subtype of HGSOC (Tothill et al., 2008), while some other markers were reported to be related to EMT in ovarian cancer or other cancers, such as SFRP4 (Ford et al., 2013), TIMP3 (Anastassiou et al., 2011), MYH11 (Y.-R. Li and W.-X. Yang, 2016) and EFEMP1 (Yin et al., 2016). Nevertheless, the link between this tumor types and a particular FTE cellular subtype was previously unrevealed. The mesenchymal subtype was previously thought to have an association with poor prognosis, but the reproducibility of the observation was inconsistent probably because of the difficulty in defining this group of tumors. Using the EMT scores from deconvolution, we reached a robust classification with consistently significant correlation with poor survival (p<0.03) in another seven independent datasets, including the AOCS dataset (Tothill et al., 2008) and six additional microarray datasets (N>100) from the CuratedOvarianData database (Ganzfried et al., 2013) (Table 3).

A DE analysis of TCGA miRNA data revealed that the miRNA-200 family (miR-200a, miR-200b, miR-200c, miR-141 and miR-429) was downregulated in EMT-high tumors (FDR<0.01, log-FC<−0.5), which agrees with the previous finding that this miRNA family suppresses EMT process and that its loss can activate EMT in invasive breast cancer cell lines with a mesenchymal phenotype (Gregory et al., 2008). We also found that miRNA-483 and miRNA-214 were significantly upregulated in EMT-high tumors, while miRNA-513c, miRNA-509 and miRNA-514 were downregulated (FIG. 4C). Although previous studies suggested that miRNA-483 and miRNA-214 play an important role in cancer progression (Chandrasekaran et al., 2016; Liu et al., 2013), their connection with EMT or ECM has not been fully studied.

TABLE 2
Patient information

| | | | | | | CELLS FOR |
|---|---|---|---|---|---|---|
| SAMPLE.ID | PATIENT.ID | SOURCE | AGE | DIAGNOSIS | TUBE STATUS | ANALYSIS |
| 11511L&R | 11511 | cryopreservation & | 69 | Endometrial cancer | | 63 |
| | | overnight cultured | | | | |
| 11519L&R | 11519 | cryopreservation & | 50 | HGSOC | | 610 |
| | | overnight cultured | | | | |
| 11528L | 11528 | cryopreservation & | 56 | HGSOC | | 258 |
| | | overnight cultured | | | | |
| 11529L | 11529 | cryopreservation & | 78 | HGSOC | | 146 |
| | | overnight cultured | | | | |
| 15062L&R | 15062 | cryopreservation & | 73 | Endometrial cancer | | 100 |
| | | overnight cultured | | | | |
| 11543L&R | 11543 | fresh | 73 | Advanced | L: Normal; | 225 |
| | | | | ovarian cancer | R: STIC | |
| 11545L&R | 11545 | fresh | 66 | Primary | Normal | 518 |
| | | | | peritoneal cancer | | |
| 15066L&R | 15066 | fresh | 52 | High-grade | Normal | 606 |
| | | | | endometrial cancer | | |
| 11553L&R | 11553 | fresh | 77 | HGSOC | L: Normal; | 464 |
| | | | | | R: mucosal carcinoma | |
| 15072L&R | 15072 | fresh | 62 | squamous cell | | 319 |
| | | | | carcinoma of | | |
| | | | | endometrium | | |
| 11553-LT | 11553 | long-term cultured | \ | \ | \ | 26 |
| 15072-LT | 15072 | long-term cultured | \ | \ | \ | 135 |
| 11553-ON | 11553 | overnight cultured | \ | \ | \ | 229 |
| 15072-ON | 15072 | overnight cultured | \ | \ | \ | 178 |

Note:
L—left tube
R—right tube
LT—long-term
ON—overnight

TABLE 3
AOCS dataset and seven microarray datasets (N > 100) from the CuratedOvarianData database

| GEO | | | HAZARD | | LOWER | UPPER | |
|---|---|---|---|---|---|---|---|
| DATABASE | SAMPLES^{ζ} | EVENTS | RATIO | P-VALUE | CI95 | CI95 | CITATION |
| E.MTAB.386 | 129 | 73 | 3.31 | 0.0120 | 1.30 | 8.41 | (Bentink |
| | | | | | | | et al., 2012) |
| GSE49997 | 194 | 57 | 3.08 | 0.0038 | 1.44 | 6.60 | (Pils et al., |
| | | | | | | | 2012) |
| GSE13876 | 157 | 113 | 3.03 | 0.0082 | 1.33 | 6.87 | (Crijns |
| | | | | | | | et al., 2009) |
| GSE26712 | 185 | 129 | 2.60 | 0.0051 | 1.33 | 5.09 | (Bonome |
| | | | | | | | et al., 2008) |
| GSE26193 | 107 | 76 | 2.42 | 0.0286 | 1.10 | 5.35 | (Mateescu |
| | | | | | | | et al., 2011) |
| GSE51088 | 152 | 112 | 1.95 | 0.0121 | 1.16 | 3.30 | (Karlan |
| | | | | | | | et al., 2014) |
| GSE32062.GPL6480 | 260 | 121 | 1.88 | 0.0556* | 0.98 | 3.58 | (Yoshihara |
| | | | | | | | et al., 2012) |
| AOCS | 253 | 106 | 2.69 | 0.0004 | 1.56 | 4.66 | (Tothill |
| (GRADE (H) | | | | | | | et al., 2008) |

^{ζ}Validation survival analysis was restricted to eight microarray datasets with over 100 samples.
*Except for GSE32062.GPL6480, the EMT scores and overall survival are negatively correlated in other seven datasets (P < 0.05). The hazard ratios of EMT scores in all eight datasets are larger than 1 (range: 1.88-3.31).

### EXAMPLE 2

To exclude the potential paracrine effect of cancer cells on non-cancer FTE cells, we validated the existence of the four secretory subtypes in the FTE cells obtained from benign (non-cancer) donors. We first analyzed 1857 single-cell transcriptomes of fallopian tubes from five patients with benign conditions (FIGS. 9A, 10A-B). Next we integrated the fresh secretory cells from the benign patients with the annotated ones from cancer patients by computing batch-correcting anchors (Stuart et al., 2019. Cell 177, 1888-1902.e21). Clustering of the integrated data illustrated that the four secretory subtypes also existed in the FTE of non-cancer donors (FIGS. 9B-D). Further validation using immunofluorescence (IF) and immunohistochemistry (IHC) in FT samples from benign donors confirmed the above results (FIGS. 10C-E). Overall, these results demonstrate that the new secretory subtypes were not artefacts caused by the influence of nearby cancer cells or by systemic effects of cancer burden.

### Example 3

According to the invention herein, a first panel of cell-signature markers was identified, as provided in Table 3 below. After further analysis, where the threshold for selecting the marker genes was adjusted, a second panel of cell-signature markers was identified, as provided in Table 4 below. Whilst both panels prove useful for identifying the cell-signatures, the second panel generated more significant (p<0.05) and reproducible results across multiple datasets.

TABLE 3
First Panel.

| | | | Entrez_ | | | KRT17 | | | |
|---|---|---|---|---|---|---|---|---|---|
| ID | HGNC_symbol | Signature | gene_id | Ensembl_gene_id | Differentiated | cluster | EMT | Cell cycle | Ciliated |
| 1 | LTBP4 | Differentiated | 8425 | ENSG00000090006 | 249.443114 | 57.3172043 | 112.25 | 103.173913 | 21.7034483 |
| 2 | PTGS1 | Differentiated | 5742 | ENSG00000095303 | 433.125749 | 238.209677 | 307.325 | 203.043478 | 20.7448276 |
| 3 | SLC25A25 | Differentiated | 114789 | ENSG00000148339 | 320.88024 | 236.344086 | 187.625 | 192.956522 | 114.358621 |
| 4 | LAMC2 | Differentiated | 3918 | ENSG00000058085 | 694.94012 | 451.069892 | 329.35 | 339.782609 | 439.137931 |
| 5 | LRG1 | Differentiated | 116844 | ENSG00000171236 | 358.568862 | 191.586022 | 203.225 | 140.173913 | 322.524138 |
| 6 | DHCR24 | Differentiated | 1718 | ENSG00000116133 | 677.347305 | 524.88172 | 319.975 | 337.130435 | 92.9241379 |
| 7 | LDLR | Differentiated | 3949 | ENSG00000130164 | 383.580838 | 245.55914 | 226.65 | 390.956522 | 35.0758621 |
| 8 | SPP1 | KRT17 Cluster | 6696 | ENSG00000118785 | 0.04191617 | 203.94086 | 0.05 | 10.9130435 | 5.56551724 |
| 9 | IL1B | KRT17 Cluster | 3553 | ENSG00000125538 | 70.3173653 | 254.069892 | 33 | 208.347826 | 7.52413793 |
| 10 | IL1RN | KRT17 Cluster | 3557 | ENSG00000136689 | 42.2335329 | 128.655914 | 21.325 | 59.826087 | 1.42068966 |
| 11 | KRT23 | KRT17 Cluster | 25984 | ENSG00000108244 | 21.9341317 | 120.349462 | 19.75 | 31.3913043 | 112.786207 |
| 12 | ALDH3B2 | KRT17 Cluster | 222 | ENSG00000132746 | 15.0718563 | 98.6935484 | 14 | 15.2608696 | 19.8827586 |
| 13 | SUSD2 | KRT17 Cluster | 56241 | ENSG00000099994 | 22.239521 | 168.704301 | 12.8 | 3.69565217 | 3.44827586 |
| 14 | DEFB1 | KRT17 Cluster | 1672 | ENSG00000164825 | 10.1916168 | 67.0752688 | 5.65 | 34.0434783 | 1.82068966 |
| 15 | HLA-DQA2 | KRT17 Cluster | 3118 | ENSG00000237541 | 7.46107784 | 61.4677419 | 20.5 | 10.7826087 | 2.06206897 |
| 16 | CYP4B1 | KRT17 Cluster | 1580 | ENSG00000142973 | 75.5868263 | 180.758065 | 57.325 | 39.6521739 | 472.606897 |
| 17 | PIGR | KRT17 Cluster | 5284 | ENSG00000162896 | 8.92814371 | 197.983871 | 23.2 | 27.6521739 | 40.0965517 |
| 18 | SPARC | EMT | 6678 | ENSG00000113140 | 12.1497006 | 12.2634409 | 234.9 | 20.7826087 | 2.11034483 |
| 19 | SERPINF1 | EMT | 5176 | ENSG00000132386 | 0.91017964 | 9.05376344 | 128.825 | 0 | 16.537931 |
| 20 | DCN | EMT | 1634 | ENSG00000011465 | 1.19760479 | 7.29569892 | 509.575 | 1.91304348 | 1.36551724 |
| 21 | SFRP4 | EMT | 6424 | ENSG00000106483 | 2.41916168 | 6.68817204 | 688.275 | 0 | 1.84827586 |
| 22 | CRISPLD2 | EMT | 83716 | ENSG00000103196 | 9.04191617 | 18.9731183 | 259.925 | 7.86956522 | 0.67586207 |
| 23 | TIMP3 | EMT | 7078 | ENSG00000100234 | 3.49700599 | 4.02688172 | 444.3 | 7.34782609 | 0.53793103 |
| 24 | CNN1 | EMT | 1264 | ENSG00000130176 | 10.0479042 | 0.49462366 | 116.625 | 6.13043478 | 10.5517241 |
| 25 | MYH11 | EMT | 4629 | ENSG00000133392 | 1.05389222 | 2.10215054 | 266.65 | 3.13043478 | 7.32413793 |
| 26 | MFAP4 | EMT | 4239 | ENSG00000166482 | 1.44311377 | 0.07526882 | 344.85 | 0 | 0.00689655 |
| 27 | ENG | EMT | 2022 | ENSG00000106991 | 3.8502994 | 3.73655914 | 103 | 0.39130435 | 3.07586207 |
| 28 | EFEMP1 | EMT | 2202 | ENSG00000115380 | 39.6706587 | 138.83871 | 273.275 | 13.0434783 | 119.427586 |
| 29 | RGS16 | EMT | 6004 | ENSG00000143333 | 2.31137725 | 2.70967742 | 259.075 | 25.173913 | 95.9931034 |
| 30 | FEN1 | Cell cycle | 2237 | ENSG00000168496 | 13.0239521 | 12.3387097 | 24.9 | 87.8695652 | 13.2344828 |
| 31 | NUSAP1 | Cell cycle | 51203 | ENSG00000137804 | 7.43712575 | 2.2688172 | 8.575 | 135.391304 | 1.82758621 |
| 32 | UBE2C | Cell cycle | 11065 | ENSG00000175063 | 0.02994012 | 0.24731183 | 2.725 | 203.043478 | 1.4137931 |
| 33 | ZWINT | Cell cycle | 11130 | ENSG00000122952 | 9.18562874 | 10.0483871 | 4.85 | 190 | 3.42758621 |
| 34 | PRC1 | Cell cycle | 9055 | ENSG00000198901 | 10.742515 | 5.19892473 | 15.675 | 122.913043 | 5.66206897 |
| 35 | ASF1B | Cell cycle | 55723 | ENSG00000105011 | 0.58083832 | 0 | 0.2 | 146.173913 | 3.84137931 |
| 36 | MCM4 | Cell cycle | 4173 | ENSG00000104738 | 42.9161677 | 28.0806452 | 65.225 | 209.608696 | 24.6275862 |
| 37 | GINS2 | Cell cycle | 51659 | ENSG00000131153 | 6.88622754 | 1.12365591 | 8.15 | 74.1304348 | 1.03448276 |
| 38 | CENPM | Cell cycle | 79019 | ENSG00000100162 | 1.19760479 | 1.44623656 | 24.575 | 77.5652174 | 80.6551724 |
| 39 | MCM2 | Cell cycle | 4171 | ENSG00000073111 | 34.9101796 | 11.9677419 | 39.325 | 92.3478261 | 33.6275862 |
| 40 | TK1 | Cell cycle | 7083 | ENSG00000167900 | 3.45508982 | 7.1344086 | 3.85 | 351.913043 | 32.7655172 |
| 41 | MCM6 | Cell cycle | 4175 | ENSG00000076003 | 14.7065868 | 8.90322581 | 53.025 | 131.26087 | 2.66896552 |
| 42 | SMC4 | Cell cycle | 10051 | ENSG00000113810 | 14.1616766 | 4.79569892 | 14.25 | 71.9565217 | 15.9931034 |
| 43 | CENPU (MLF1IP) | Cell cycle | 79682 | ENSG00000151725 | 1.75449102 | 1.30645161 | 8.725 | 71.173913 | 4.19310345 |
| 44 | MAD2L1 | Cell cycle | 4085 | ENSG00000164109 | 8.0239521 | 4.31182796 | 3 | 87.173913 | 37.0344828 |
| 45 | TEKT1 | Ciliated | 83659 | ENSG00000167858 | 5.5988024 | 0.8655914 | 0.3 | 0.82608696 | 800 |
| 46 | FAM92B | Ciliated | 339145 | ENSG00000153789 | 0.02994012 | 0.05913978 | 0 | 0 | 589.331034 |
| 47 | SNTN | Ciliated | 132203 | ENSG00000188817 | 1.03592814 | 4.53225806 | 0 | 2.47826087 | 800 |
| 48 | LRRC46 | Ciliated | 90506 | ENSG00000141294 | 2.08982036 | 3.40860215 | 5.075 | 1.17391304 | 501.317241 |
| 49 | EFCAB1 | Ciliated | 79645 | ENSG00000034239 | 0.98203593 | 0.79032258 | 0 | 0.04347826 | 611.6 |
| 50 | CDHR3 | Ciliated | 222256 | ENSG00000128536 | 1.51497006 | 2.12365591 | 0 | 0 | 659.655172 |
| 51 | C6orf118 | Ciliated | 168090 | ENSG00000112539 | 17.7125749 | 2.11827957 | 0 | 14.3913043 | 493.765517 |
| 52 | CCDC78 | Ciliated | 124093 | ENSG00000162004 | 0.04790419 | 0.19354839 | 0 | 0 | 702.027586 |

The table lists 52 marker genes.
The HGNC gene symbol is listed in the second column, the Entrez gene ID in the fourth column and the Ensembl gene ID in the fifth column.
The third column describes which signature the gene belongs to.
The sixth to tenth columns show the scaled expression levels of each gene in a certain cell state signature.
The numbers are used in the deconvolution step to calculate the cell state proportions.

TABLE 4
Second Panel.

| | | | Entrez_ | | | KRT17 | | | |
|---|---|---|---|---|---|---|---|---|---|
| ID | HGNC_symbol | Signature | gene_id | Ensembl_gene_id | Differentiated | cluster | EMT | Cell cycle | Ciliated |
| 1 | LTBP4 | Differentiated | 8425 | ENSG00000090006 | 249.443114 | 57.3172043 | 112.25 | 103.173913 | 21.7034483 |
| 2 | SLC25A25 | Differentiated | 114789 | ENSG00000148339 | 320.88024 | 236.344086 | 187.625 | 192.956522 | 114.358621 |
| 3 | LAMC2 | Differentiated | 3918 | ENSG00000058085 | 694.94012 | 451.069893 | 329.35 | 339.782609 | 439.137931 |
| 4 | DHCR24 | Differentiated | 1718 | ENSG00000116133 | 677.347305 | 524.88172 | 319.975 | 337.130435 | 92.9241379 |
| 5 | PLK3 | Differentiated | 1263 | ENSG00000173846 | 151.700599 | 111.655914 | 78.725 | 106.608696 | 65.6275862 |
| 6 | LRG1 | Differentiated | 116844 | ENSG00000171236 | 358.568862 | 191.586022 | 203.225 | 140.173913 | 322.524138 |
| 7 | LDLR | Differentiated | 3949 | ENSG00000130164 | 383.580838 | 245.55914 | 226.65 | 390.956522 | 35.0758621 |
| 8 | SPP1 | KRT17 Cluster | 6696 | ENSG00000118785 | 0.04191617 | 203.94086 | 0.05 | 10.9130435 | 5.56551724 |
| 9 | IL1B | KRT17 Cluster | 3553 | ENSG00000125538 | 70.3173653 | 254.069893 | 33 | 208.347826 | 7.52413793 |
| 10 | IL1RN | KRT17 Cluster | 3557 | ENSG00000136689 | 42.2335329 | 128.655914 | 21.325 | 59.826087 | 1.42068966 |
| 11 | KRT23 | KRT17 Cluster | 25984 | ENSG00000108244 | 21.9341317 | 120.349462 | 19.75 | 31.3913044 | 112.786207 |
| 12 | ALDH3B2 | KRT17 Cluster | 222 | ENSG00000132746 | 15.0718563 | 98.6935484 | 14 | 15.2608696 | 19.8827586 |
| 13 | SUSD2 | KRT17 Cluster | 56241 | ENSG00000099994 | 22.239521 | 168.704301 | 12.8 | 3.69565217 | 3.44827586 |
| 14 | DEFB1 | KRT17 Cluster | 1672 | ENSG00000164825 | 10.1916168 | 67.0752688 | 5.65 | 34.0434783 | 1.82068966 |
| 15 | HLA-DQA2 | KRT17 Cluster | 3118 | ENSG00000237541 | 7.46107784 | 61.4677419 | 20.5 | 10.7826087 | 2.06206897 |
| 16 | CYP4B1 | KRT17 Cluster | 1580 | ENSG00000142973 | 75.5868264 | 180.758065 | 57.325 | 39.6521739 | 472.606897 |
| 17 | PIGR | KRT17 Cluster | 5284 | ENSG00000162896 | 8.92814371 | 197.983871 | 23.2 | 27.6521739 | 40.0965517 |
| 18 | SPARC | EMT | 6678 | ENSG00000113140 | 12.1497006 | 12.2634409 | 234.9 | 20.7826087 | 2.11034483 |
| 19 | SERPINF1 | EMT | 5176 | ENSG00000132386 | 0.91017964 | 9.05376344 | 128.825 | 0 | 16.537931 |
| 20 | DCN | EMT | 1634 | ENSG00000011465 | 1.19760479 | 7.29569893 | 509.575 | 1.91304348 | 1.36551724 |
| 21 | SFRP4 | EMT | 6424 | ENSG00000106483 | 2.41916168 | 6.68817204 | 688.275 | 0 | 1.84827586 |
| 22 | CRISPLD2 | EMT | 83716 | ENSG00000103196 | 9.04191617 | 18.9731183 | 259.925 | 7.86956522 | 0.67586207 |
| 23 | TIMP3 | EMT | 7078 | ENSG00000100234 | 3.49700599 | 4.02688172 | 444.3 | 7.34782609 | 0.53793103 |
| 24 | CNN1 | EMT | 1264 | ENSG00000130176 | 10.0479042 | 0.49462366 | 116.625 | 6.13043478 | 10.5517241 |
| 25 | MYH11 | EMT | 4629 | ENSG00000133392 | 1.05389222 | 2.10215054 | 266.65 | 3.13043478 | 7.32413793 |
| 26 | MFAP4 | EMT | 4239 | ENSG00000166482 | 1.44311377 | 0.07526882 | 344.85 | 0 | 0.00689655 |
| 27 | ENG | EMT | 2022 | ENSG00000106991 | 3.8502994 | 3.73655914 | 103 | 0.39130435 | 3.07586207 |
| 28 | EFEMP1 | EMT | 2202 | ENSG00000115380 | 39.6706587 | 138.83871 | 273.275 | 13.0434783 | 119.427586 |
| 29 | RGS16 | EMT | 6004 | ENSG00000143333 | 2.31137725 | 2.70967742 | 259.075 | 25.173913 | 95.9931035 |
| 30 | FEN1 | Cell cycle | 2237 | ENSG00000168496 | 13.0239521 | 12.3387097 | 24.9 | 87.8695652 | 13.2344828 |
| 31 | NUSAP1 | Cell cycle | 51203 | ENSG00000137804 | 7.43712575 | 2.2688172 | 8.575 | 135.391304 | 1.82758621 |
| 32 | UBE2C | Cell cycle | 11065 | ENSG00000175063 | 0.02994012 | 0.24731183 | 2.725 | 203.043478 | 1.4137931 |
| 33 | ZWINT | Cell cycle | 11130 | ENSG00000122952 | 9.18562874 | 10.0483871 | 4.85 | 190 | 3.42758621 |
| 34 | PRC1 | Cell cycle | 9055 | ENSG00000198901 | 10.742515 | 5.19892473 | 15.675 | 122.913044 | 5.66206897 |
| 35 | ASF1B | Cell cycle | 55723 | ENSG00000105011 | 0.58083832 | 0 | 0.2 | 146.173913 | 3.84137931 |
| 36 | MCM4 | Cell cycle | 4173 | ENSG00000104738 | 42.9161677 | 28.0806452 | 65.225 | 209.608696 | 24.6275862 |
| 37 | GINS2 | Cell cycle | 51659 | ENSG00000131153 | 6.88622755 | 1.12365591 | 8.15 | 74.1304348 | 1.03448276 |
| 38 | CENPM | Cell cycle | 79019 | ENSG00000100162 | 1.19760479 | 1.44623656 | 24.575 | 77.5652174 | 80.6551724 |
| 39 | MCM2 | Cell cycle | 4171 | ENSG00000073111 | 34.9101796 | 11.9677419 | 39.325 | 92.3478261 | 33.6275862 |
| 40 | TK1 | Cell cycle | 7083 | ENSG00000167900 | 3.45508982 | 7.1344086 | 3.85 | 351.913044 | 32.7655172 |
| 41 | MCM6 | Cell cycle | 4175 | ENSG00000076003 | 14.7065868 | 8.90322581 | 53.025 | 131.26087 | 2.66896552 |
| 42 | SMC4 | Cell cycle | 10051 | ENSG00000113810 | 14.1616767 | 4.79569893 | 14.25 | 71.9565217 | 15.9931035 |
| 43 | CENPU (MLF1IP) | Cell cycle | 79682 | ENSG00000151725 | 1.75449102 | 1.30645161 | 8.725 | 71.173913 | 4.19310345 |
| 44 | MAD2LI | Cell cycle | 4085 | ENSG00000164109 | 8.0239521 | 4.31182796 | 3 | 87.173913 | 37.0344828 |
| 45 | TEKT1 | Ciliated | 83659 | ENSG00000167858 | 5.5988024 | 0.8655914 | 0.3 | 0.82608696 | 760.510552 |
| 46 | TUBA4B | Ciliated | 80086 | ENSG00000243910 | 0.13772455 | 1.17741936 | 0.05 | 0.26086957 | 674.096552 |
| 47 | C20orf85 | Ciliated | 128602 | ENSG00000124237 | 0.83233533 | 2.70967742 | 0 | 0 | 760.510552 |
| 48 | CAPSL | Ciliated | 133690 | ENSG00000152611 | 4.94610778 | 5.50537634 | 6.775 | 0.04347826 | 760.510552 |
| 49 | LRRC46 | Ciliated | 90506 | ENSG00000141294 | 2.08982036 | 3.40860215 | 5.075 | 1.17391304 | 501.317241 |
| 50 | EFCAB1 | Ciliated | 79645 | ENSG00000034239 | 0.98203593 | 0.79032258 | 0 | 0.04347826 | 611.6 |
| 51 | C6orf118 | Ciliated | 168090 | ENSG00000112539 | 17.7125749 | 2.11827957 | 0 | 14.3913044 | 493.765517 |
| 52 | CCDC78 | Ciliated | 124093 | ENSG00000162004 | 0.04790419 | 0.19354839 | 0 | 0 | 702.027586 |

The table lists 52 marker genes.
The HGNC gene symbol is listed in the second column, the Entrez gene 10 in the fourth column and the Ensembl gene ID in the fifth column.
The third column describes which signature the gene belongs to.
The sixth to tenth columns show the scaled expression levels of each gene in a certain cell state signature.
The numbers are used in the deconvolution step to calculate the cell state proportions.

Comparison between the First Panel and the Second Panel

By comparing the survival analysis results, the second gene panel generated more significant (p<0.05) and reproducible results across multiple datasets.

TABLE 5
Multivariate survival analysis of patients' overall survival against
EMT scores, grades and stages by using the first panel in the deconvolution analysis

| | N | N | Hazard | | Lower | Upper |
|---|---|---|---|---|---|---|
| Dataset | samples | events | ratio | p | CI95 | CI95 |
| E.MTAB.386 | 128 | 73 | 2.4 | 0.056 | 1.0 | 5.7 |
| GSE13876 | 144 | 105 | 1.8 | 0.119 | 0.9 | 3.9 |
| GSE26193 | 79 | 60 | 2.3 | 0.057 | 1.0 | 5.5 |
| GSE26712 | 185 | 129 | 2.0 | 0.040 | 1.0 | 3.7 |
| GSE32062.GPL6480 | 260 | 121 | 1.8 | 0.067 | 1.0 | 3.5 |
| GSE49997 | 170 | 47 | 2.5 | 0.049 | 1.0 | 6.1 |
| GSE51088 | 113 | 93 | 2.4 | 0.010 | 1.2 | 4.8 |
| TCGA | 184 | 307 | 2.2 | 0.011 | 1.2 | 4.0 |
| AOCS | 109 | 253 | 2.2 | 0.005 | 1.3 | 3.9 |

TABLE 6
Multivariate survival analysis of patients' overall survival against
EMT scores, grades and stages by using the second panel in the deconvolution analysis

| | N | N | Hazard | | Lower | Upper |
|---|---|---|---|---|---|---|
| Dataset | samples | events | ratio | p | CI95 | CI95 |
| E.MTAB.386 | 128 | 73 | 3.1 | 0.019 | 1.2 | 7.9 |
| GSE13876 | 144 | 105 | 2.9 | 0.013 | 1.3 | 6.7 |
| GSE26193 | 79 | 60 | 2.6 | 0.041 | 1.0 | 6.5 |
| GSE26712 | 185 | 129 | 2.0 | 0.031 | 1.1 | 3.9 |
| GSE32062.GPL6480 | 260 | 121 | 1.9 | 0.054 | 1.0 | 3.6 |
| GSE49997 | 170 | 47 | 2.8 | 0.022 | 1.2 | 6.9 |
| GSE51088 | 113 | 93 | 2.1 | 0.022 | 1.1 | 3.9 |
| TCGA | 184 | 307 | 2.2 | 0.009 | 1.2 | 3.9 |
| AOCS | 109 | 253 | 2.1 | 0.008 | 1.2 | 3.7 |

### EXAMPLE 4

### The Immunophenotype

We investigated if the EMT scores correlate with the immunophenotype of SOC. We computed the proportion of multiple types of leukocytes in the TCGA data by using CIBERSORT. We used both the LM22 and LM6 signatures, which generates two sets of deconvolution results. In the results generated by using LM22, the EMT-high tumors have significantly higher proportion of macrophage M2 (FIG. 11A). In the results generated by using LM6, the EMT-high tumors have significantly higher proportion of monocytes (FIG. 11B). We next conducted association analysis between the EMT scores and the expression levels of macrophage marker genes, which shows that the positive correlation also existed between the EMT scores and the expression of macrophage markers (FIG. 11C). Overall, the results suggest that there is a positive association between macrophages M2 and EMT components in serous ovarian tumours. Therefore, the method of determining the status of high-grade serous ovarian carcinoma (HGSOC) in a subject could indicate that immunotherapy can be used to target the tumour.

### REFERENCES

All references cited herein are incorporated by reference.

Ansieau, S. et al. Cancer Cell 14, 79-89. doi:10.1016/j.ccr.2008.06.005

Baron, M., et al., 2016. Cell Syst 3, 346-. doi:10.1016/j.cels.2016.08.011

Bell, D. et al. 2011. Integrated genomic analyses of ovarian carcinoma. Nature 474, 609-615. doi:10.1038/nature10166

Campbell, K. R., Yau, C., 2018. Nat Commun 9. doi:10.1038/s41467-018-04696-6

Cerny, K. L., et al. PLoS ONE 11, e0147685. doi:10.1371/journal.pone.0147685

Chandrasekaran, K. S. et al. 2016. Br. J. Cancer 115, 741-751. doi: 10.1038/bjc.2016.234

Chen, G. M., et al. 2018. Clin. Cancer Res. 24, 5037-5047. doi:10.1158/1078-0432.CCR-18-0784

Comer, M. T., et al. 1998. Human Reproduction 13, 3114-3120. doi:10.1093/humrep/13.11.3114

Fan, J., et al. 2018. Linking transcriptional and genetic tumor heterogeneity through allele analysis of single-cell RNA-seq data. Genome Res. gr.228080.117. doi:10.1101/gr.228080.117

Ford, C. E., et al. 2013. PLoS ONE 8, e54362. doi:10.1371/journal.pone.0054362

Fresno Vara, J. A., et al. 2004. Cancer Treat. Rev. 30, 193-204. doi: 10.1016/j .ctrv.2003 .07.007

Ganzfried, B. F., et al. 2013. CuratedOvarianData: clinically annotated data for the ovarian cancer transcriptome. Database (Oxford) 2013, bat013. doi:10.1093/database/bat013

Ghosh, A., et al. 2017. Development 144, 3031-3041. doi:10.1242/dev.149989

Gregory, P. A., et al. 2008. Nat. Cell Biol. 10, 593-601. doi:10.1038/ncb1722

Hanahan, D., Weinberg, R. A., 2011. Hallmarks of Cancer: The Next Generation. Cell 144, 646-674. doi:10.1016/j.ce11.2011.02.013

Hellner, K., et al. 2016EBIOM 10, 137-149. doi:10.1016/j.ebiom.2016.06.048

Honkaniemi, J. et al. 1992. Neuroreport 3, 849-852.

Kang, Y., Massagué, J., 2004. Cell 118, 277-279. doi:10.1016/j.ce11.2004.07.011

Klos Dehring, D. A., et al. 2013. Dev. Cell 27, 103-112. doi: 10.1016/j .devce1.2013.08.021

Kuhn, E., et al. 2012. International Journal of Gynecological Pathology 31, 416-422. doi:10.1097/PGP.0b013e31824cbeb4

Li, Y.-R., Yang, W.-X., 2016. Oncotarget 7, 46785-46812. doi:10.18632/oncotarget.8800

Liu, M., et al. 2013. Genes Dev. 27, 2543-2548. doi:10.1101/gad.224170.113

Mahalingaiah, P. K. S., Ponnusamy, L., Singh, K. P., 2015. Cells. J. Cell. Physiol. 230, 1916-1928. doi:10.1002/jcp.24922

McLean, G. W., et al. 2005. Nature Reviews Cancer 5, 505-515. doi:10.1038/nrc1647

Nalepa, G., Clapp, D. W., 2018. Nature Reviews Cancer 18, 168-185. doi:10.1038/nrc.2017.116

Newman, A. M., et al. 2015. Nat Meth 12, 453-. doi:10.1038/NMETH.3337

Paik, D. Y., et al. 2012. Stem Cells 30, 2487-2497. doi:10.1002/stem.1207

Picelli, S., et al. 2014. Nature Protocols 9, 171-181. doi:10.1038/nprot.2014.006

Wang, S., et al. 2002. Biochem. Biophys. Res. Commun. 291, 414-420. doi:10.1006/bbrc.2002.6461

Wu, R., et al. 2016. J. Pathol. 240, 341-351. doi:10.1002/path.4783

Yang, J., et al. 2004. Cell 117, 927-939. doi:10.1016/j.ce11.2004.06.006

Yin, X., et al. 2016. Oncotarget 7, 47938-47953. doi:10.18632/oncotarget.10296

Bentink, S., et al. 2012. PLoS ONE 7, e30269. doi:10.1371/journal.pone.0030269

Bonome, T., et al. 2008. Cancer Res. 68, 5478-5486. doi:10.1158/0008-5472.CAN-07-6595

Crijns, A. P. G., et al. 2009. PLoS Med. 6, e24. doi:10.1371/journal.pmed.1000024

Karlan, B. Y., et al. 2014. Gynecol. Oncol. 132, 334-342. doi:10.1016/j.ygyno.2013.12.021

Mateescu, B., et al. 2011. Nat. Med. 17, 1627-1635. doi:10.1038/nm.2512

Pils, D., et al. 2012. Cancer Sci. 103, 1334-1341. doi:10.1111/j.1349-7006.2012.02306.x

Tothill, R. W., Tinker, A. V., George, J., Brown, R., Fox, S. B., Lade, S., Johnson, D. S.,

Trivett, M. K., Etemadmoghadam, D., Locandro, B., Traficante, N., Fereday, S., Hung, J. A., Chiew, Y.-E., Haviv, I., Australian Ovarian Cancer Study Group, Gertig, D., DeFazio, A., Bowtell, D. D. L., 2008. Novel molecular subtypes of serous and endometrioid ovarian cancer linked to clinical outcome. Clin. Cancer Res. 14, 5198-5208. doi:10.1158/1078-0432.CCR-08-0196

Yoshihara, K., Tsunoda, T., Shigemizu, D., Fujiwara, H., Hatae, M., Fujiwara, H., Masuzaki, H., Katabuchi, H., Kawakami, Y., Okamoto, A., Nogawa, T., Matsumura, N., Udagawa, Y., Saito, T., Itamochi, H., Takano, M., Miyagi, E., Sudo, T., Ushijima, K., Iwase, H., Seki, H., Terao, Y., Enomoto, T., Mikami, M., Akazawa, K., Tsuda, H., Moriya, T., Tajima, A., Inoue, I., Tanaka, K., Japanese Serous Ovarian Cancer Study Group, 2012. High-risk ovarian cancer based on 126-gene expression signature is uniquely characterized by downregulation of antigen presentation pathway. Clin. Cancer Res. 18, 1374-1385. doi:10.1158/1078-0432.CCR-11-2725

1. A method of determining the status of high-grade serous ovarian carcinoma (HGSOC) in a subject, the method comprising:
providing a sample obtained from the subject; and
detecting the presence of HGSOC biomarkers in the sample, wherein the method comprises detecting the presence of:
a differentiated cell type by detecting one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
a KRT17 Cluster cell type by detecting one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
an epithelial-mesenchymal transition (EMT) cell type by detecting one or more of epithelial-mesenchymal transition (EMT) biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
a cell cycle cell type by detecting one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
a ciliated cell type by detecting one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL;
wherein the level of the biomarkers is used to determine the fraction of EMT cells in the high-grade serous ovarian carcinoma in the subject.

2. The method according to claim 1, wherein the fraction of EMT cells in the high-grade serous ovarian carcinoma in the subject is compared to a pre-determined threshold level to indicate if the high-grade serous ovarian carcinoma in the subject is an EMT subclass of high-grade serous ovarian carcinoma.

3. The method according to claim 1 or claim 2, wherein the level of the EMT biomarkers relative to the differentiated, KRT17 Cluster, cell cycle and ciliated biomarkers is indicative of the fraction of EMT cells, and the fraction of EMT cells above a pre-determined threshold level is indicative of an EMT subclass of high-grade serous ovarian carcinoma in the subject.

4. A method of detecting a panel of biomarkers in a sample of a subject, the method comprising:
providing a sample obtained from a subject and detecting the presence of:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

5. The method according to any preceding claim, wherein the nucleic acid encoding the biomarker comprises mRNA transcripts, or cDNA copies thereof, of the biomarkers.

6. The method according to any preceding claim, wherein detecting the level of a biomarker comprises the use of an oligonucleotide probe capable of binding to nucleic acid encoding the biomarker.

7. The method according to any preceding claim, wherein the method comprises determining the transcript level of the biomarkers.

8. The method according to any preceding claim, wherein the sample from the subject is ovarian cancer biopsy tissue.

9. The method according to any preceding claim, wherein all 52 of the biomarkers are detected.

10. A composition comprising a panel of probes, wherein the probes are for detecting:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

11. A kit for determining the status of high-grade serous ovarian carcinoma (HGSOC) in a subject, the kit comprising a panel of probes, wherein the probes are for detecting:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

12. The composition according claim 10, or the kit according to claim 11, wherein the panel of probes comprises probes for all 52 of the biomarkers.

13. A method of selecting a patient for treatment with an agent, agent combination, or composition for treatment or prevention of HGSOC, the method comprising determining the status of high-grade serous ovarian carcinoma (HGSOC) in a subject according to the method of any of claims 1-3 and 5-9, wherein the determination of an EMT subclass of HGSOC indicates that the subject should or should not receive the agent, agent combination, or composition.

14. A PI3K pathway inhibitor and/or immunotherapeutic agent for use in the treatment of high-grade serous ovarian carcinoma (HGSOC) in a subject, wherein the treatment comprises selecting the patient for treatment based on the determination of an EMT subclass of high-grade serous ovarian carcinoma in the subject according to the method of any of claims 1-3 and 5-9.

15. A method of treatment of high-grade serous ovarian carcinoma, wherein the subject is determined to have an EMT subclass of high-grade serous ovarian carcinoma according to the method of any of claims 1-3 and 5-9;
wherein the method of treatment comprises administrating a PI3K pathway inhibitor and/or immunotherapeutic agent to the subject.

16. A method of treating a high-grade serous ovarian carcinoma in a subject with, the method comprising the steps of:
receiving results of a biomarker assay of a tissue sample from the subject to determine if the patient has an EMT subclass of high-grade serous ovarian carcinoma; and
if the subject has an EMT subclass of high-grade serous ovarian carcinoma, then administrating a PI3K pathway inhibitor and/or immunotherapeutic agent to the subject,
wherein the biomarker assay is in accordance with the method of any of claims 1-3 and 5-9.

17. Use of a panel of biomarkers for determining the fraction of EMT cells present in a tissue sample from a subject with HGSOC, or for determining the status of a HGSOC in a subject, wherein the biomarkers comprise:
one or more of differentiated cell biomarker proteins and/or nucleic acid encoding differentiated cell biomarker proteins selected from the group comprising LTBP4, PTGS1, SLC25A25, LAMC2, LRG1, DHCR24, PLK3 and LDLR;
one or more of KRT17 Cluster cell biomarker proteins and/or nucleic acid encoding KRT17 Cluster cell biomarker proteins selected from the group comprising SPP1, IL1B, IL1RN, KRT23, ALDH3B2, SUSD2, DEFB1, HLA-DQA2, CYP4B1, and PIGR;
one or more of EMT biomarker proteins and/or nucleic acid encoding EMT biomarker proteins selected from the group comprising SPARC, SERPINF1, DCN, SFRP4, CRISPLD2, TIMP3, CNN1, MYH11, MFAP4, ENG, EFEMP1, and RGS16;
one or more of cell cycle biomarker proteins and/or nucleic acid encoding cell cycle biomarker proteins selected from the group comprising FEN1, NUSAP1, UBE2C, ZWINT, PRC1, ASF1B, MCM4, GINS2, CENPM, MCM2, TK1, MCM6, SMC4, CENPU, and MAD2L1; and
one or more of ciliated cell biomarker proteins and/or nucleic acid encoding ciliated cell biomarker proteins selected from the group comprising TEKT1, FAM92B, SNTN, LRRC46, EFCAB1, CDHR3, C6orf118, CCDC78, TUBA4B, C20orf85 and CAPSL.

18. The use according to claim 17, wherein the use comprise the use of the composition of claim 10 or 12, or the kit of claim 11 or 12.
